(19) United States
(12) Patent Application Publication
(10) Pub. No.: US 2026/0341453 A1
(43) Pub. Date: Oct. 29, 2026

(54) SYSTEMS AND METHODS FOR MEDICATION AND DISEASE MANAGEMENT

(71) Applicant: Sanofi, Paris (FR)

(72) Inventors: Niranjan TUMMINKATTI, Cambridge, MA (US); Jesse ALVAREZ, Cambridge, MA (US); Veronique BRUHAT, Paris (FR); Caroline LANZONI MCCRAY, Cambridge, MA (US); Junqian LIANG, Cambridge, MA (US); Marina Martha TALAVARES FLORES, Barcelona (ES)

(21) Appl. No.: 19/123,084

(22) PCT Filed: Oct. 24, 2023
(86) PCT No.: PCT/IB2023/000624

§ 371 Date: Apr. 22, 2025

Related U.S. Application Data
(60) Provisional application No. 63/418,734, filed on Oct. 24, 2022.

(30) Foreign Application Priority Data
Mar. 28, 2023 (EP) 23315065.5

(51) Int. Cl. G16H 20/13; G16H 40/67; G16H 80/00

(52) U.S. Cl. CPC G16H 20/13; G16H 40/67; G16H 80/00

(57) ABSTRACT

A non-transitory computer readable storage medium, user device and method configured to execute a program for managing a plurality of medical conditions being treated by a first medicament; provide a user interface allowing a user to specify a first medical condition and a second medical condition being treated by the first medicament; provide treatment access information related to the first medicament; provide at least one of: information relating to the first medical condition, a journal facilitating input of symptoms related to the first medical condition and a notification relating to the first medical condition; and provide at least one of information relating to the second medical condition, a journal facilitating input of symptoms related to the second medical condition, a notification relating to the second medical condition.

### CROSS REFERENCE TO RELATED APPLICATIONS

The present application is the national stage entry of International Patent Application No. PCT/IB2023/000624, filed on Oct. 24, 2023, and claims priority to Application No. EP 23315065.5, filed on Mar. 28, 2023, and U.S. Application No. 63/418,734, filed Oct. 24, 2022, the disclosures of which are incorporated herein by reference.

### TECHNICAL FIELD

The present specification relates to systems and methods for medication and disease management and, in particular, to a computer implemented system and method for assisting a user to manage one or more diseases capable of being treated by one or more medicaments.

### BACKGROUND

A significant barrier to high quality therapeutic outcomes is patient engagement and adherence with a prescribed medication regimen as well as patient understanding of the factors affecting their disease progression and overall health.

Current systems do not effectively allow patients with complex morbidities to manage multiple health conditions in a convenient way. Current systems also do not effectively allow patients to track the progress of multiple health conditions or facilitate access to information and guidance relating to these conditions.

### SUMMARY

Certain aspects of this disclosure relate to systems and methods to provide effective and informative assistance to patients in managing their health conditions, which improve patient engagement and adherence and ultimately leads to improved therapeutic outcomes for the patient.

A first aspect of this specification provides a computer implemented method, the method comprising:
executing a program for managing a plurality of medical conditions being treated by a first medicament;
providing a user interface allowing a user to specify a first medical condition being treated by the first medicament;
providing a user interface allowing a user to specify a second medical condition being treated by the first medicament;
providing treatment access information related to the first medicament;
providing at least one of: information relating to the first medical condition, a journal facilitating input of symptoms related to the first medical condition and a notification relating to the first medical condition; and
providing at least one of information relating to the second medical condition, a journal facilitating input of symptoms related to the second medical condition, a notification relating to the second medical condition.

Providing the user interface may comprise providing a single user interface display allowing the user to specify both the first medical condition being treated by the first medicament and the second medical condition being treated by the first medicament. The user interface may allow a user to specify further medicaments being used to treat the first medical condition, second medical condition or a further medical condition. In a particular example, the first medical condition is Atopic Dermatitis and the second medical condition is Asthma.

The method may further comprise pairing the program for managing a plurality of medical conditions being treated by a first medicament with one or more external devices and receiving user specific data from the one or more external devices. The user specific data may be physiological data and/or activity data.

The method may further comprise receiving environmental information and displaying the environmental information.

The method may further comprise determining that an authorization is required to customize the information relating to the first medical condition and/or the information relating to the second medical condition; and presenting a user interface to obtain the authorization. The authorization may be to access the user's location.

The method may further comprise providing a dose support screen having a call-scheduling element configured to initiate a call booking process with a healthcare professional when selected. The method may further comprise providing a pre-call instruction screen containing: instructions for preparing and/or administering the first medicament; and a call-initiating element configured to initiate a call be opening a phone application when selected. The method may further comprise providing a post-call screen containing a body representation with multiple predefined areas for indicating the location of an injection.

The method may further comprise providing a reminder system configured to: allow input of medication administration reminder parameters; and generate a reminder for display on a graphical user interface provided by the program for managing a plurality of medical conditions being treated by a first medicament or for display as a push notification on a locked screen of a user device.

The method may further comprise providing a prescription management system configured to: allow input of collection or delivery details for the first medicament; display status updates relating to collection or delivery details of the first medicament; and generate one or more push notifications relating to collection or delivery details of the first medicament.

The method may further comprise: providing a user interface facilitating the input of insurance and/or financial information related to accessing the first medicament; sending the insurance and/or financial information to a third party; and displaying an insurance and/or financial status notification related to accessing the first medicament.

The method may further comprise: scanning a code on an injection device in order to check an expiry date of medicament within the injection device; and when it is determined that the medicament within the injection device is expired, outputting a warning via the program.

The method may further comprise: scanning a code associated with a new injection device or a new batch of injection devices; launching the program and determining that the user is a registered user of the program; and displaying a user interface asking the user to confirm that they have received a new injection device or batch of injection devices.

The method may further comprise, in response to receiving a user input confirming that they have received the new injection device or batch of injection devices, causing a refill reminder in the program to be reset.

A second aspect of this specification provides a non-transitory computer readable storage medium comprising instructions that, when executed by a computer, cause the computer to:
execute a program for managing a plurality of medical conditions being treated by a first medicament;
provide a user interface allowing a user to specify a first medical condition being treated by the first medicament;
provide a user interface allowing a user to specify a second medical condition being treated by the first medicament;
provide treatment access information related to the first medicament;
provide at least one of: information relating to the first medical condition, a journal facilitating input of symptoms related to the first medical condition and a notification relating to the first medical condition; and
provide at least one of information relating to the second medical condition, a journal facilitating input of symptoms related to the second medical condition, a notification relating to the second medical condition.

The instructions may cause the computer to provide a single user interface display allowing the user to specify both the first medical condition being treated by the first medicament and the second medical condition being treated by the first medicament.

The instructions may cause the computer to: determine that an authorization is required to customize the information relating to the first medical condition and/or the information relating to the second medical condition; and present a user interface to obtain the authorization. The authorization may be to access the user's location.

The instructions may cause the computer to receive environmental information and display the environmental information.

A third aspect of this specification provides a user device comprising processing circuitry configured to:
execute a program for managing a plurality of medical conditions being treated by a first medicament;
provide a user interface allowing a user to specify a first medical condition being treated by the first medicament;
provide a user interface allowing a user to specify a second medical condition being treated by the first medicament;
provide treatment access information related to the first medicament;
provide at least one of: information relating to the first medical condition, a journal facilitating input of symptoms related to the first medical condition and a notification relating to the first medical condition; and
provide at least one of information relating to the second medical condition, a journal facilitating input of symptoms related to the second medical condition, a notification relating to the second medical condition.

The processing circuitry may be further configured to pair the program for managing a plurality of medical conditions being treated by a first medicament with one or more external devices and receive user specific data from the one or more external devices. The user specific data may be physiological data and/or activity data.

The processing circuitry may be further configured to: control a camera of the user device to scan a code on an injection device; check an expiry date of medicament within the injection device based on the scanned code; and when it is determined that the medicament within the injection device is expired, output a warning via the program.

The processing circuitry may be further configured to: control a camera of the user device to scan a code associated with a new injection device or a new batch of injection devices; launch the program and determine that the user is a registered user of the program; and display a user interface asking the user to confirm that they have received a new injection device or batch of injection devices. The processing circuitry may be further configured to, in response to receiving a user input confirming that they have received the new injection device or batch of injection devices, cause a refill reminder in the program to be reset.

### BRIEF DESCRIPTION OF THE FIGURES

So that the general concepts set out in the foregoing sections can be more fully understood, embodiments thereof will be described with reference to the accompanying drawings, in which:

FIG. 1a shows an overall architecture of a system in which the computer program operates;

FIG. 1b shows a flowchart illustrating the overall process of user account creation and login;

FIGS. 1c to 1k show screenshots illustrating a user account creation process;

FIGS. 2a to 2m show screenshots illustrating an application customization process;

FIG. 2n shows a data flow diagram for the application customization process;

FIG. 3a shows a dose support screen of the application;

FIG. 3b shows a user interface for inputting user contact preferences;

FIG. 4a shows an example of a push notification for an upcoming call;

FIG. 4b shows an instruction screen for the upcoming call;

FIG. 4c shows an injection location screen displayed after the call;

FIG. 5 shows an example of a home screen of the application;

FIG. 6 shows an example of a user profile in the application;

FIG. 7 shows an injection routine control screen;

FIG. 8 shows an example of a graphical user interface comprising in-application notification prompting the user to make a journal entry;

FIG. 9 shows a push notification to a locked screen of a user device prompting the user to make a journal entry;

FIG. 10a shows a health condition entry screen of the application;

FIG. 10b shows a photograph entry screen;

FIG. 10c shows an activity tracker data selection screen;

FIG. 11 is a data flow diagram illustrating categories of data and movement of data;

FIG. 12a shows a treatments tab screen;

FIGS. 12b and 12c show a report screen;

FIG. 13a shows an exemplary journal tab screen;

FIG. 13b shows an alternative journal tab screen;

FIG. 14 shows an exemplary learning tab screen;

FIG. 15a shows an exemplary account tab screen;

FIGS. 15b and 15c show a settings tab screen;

FIG. 16 illustrates environmental information;

FIG. 17 shows user physiological and activity data;

FIGS. 18a and 18b show how the environmental information of FIG. 16 and user physiological and activity data of FIG. 17 may be displayed within the application;

FIGS. 19a to 19c show how the application assists the user with medicament collection or delivery;

FIGS. 20a to 20c show how the application assists the user with linking their insurance details with the application and/or making an insurance claim;

FIG. 21 illustrates schematically a user device for running the computer program; and

FIG. 22 is a flow chart illustrating the steps of a computer implemented method.

### DETAILED DESCRIPTION

The present disclosure relates to an application which can be run on a user device and which assists a user with managing their multiple health conditions, as well as facilitating access to medicaments, learning resources and financial guidance relating to a medicament. The application provides a wide range of resources and functionality to assist the user in a single program. For example, the application provides a customized onboarding process in relation to two or more diseases/health conditions so that it can subsequently present information and provide notifications in relation to these health conditions as well as providing a journal for entering symptoms and tracking the disease progression and treatment regimen in relation to these health conditions. The application also assists the user in managing appointments and calls with healthcare professionals, proving appropriate reminders and summary reports. The application also assists the user in obtaining their medicament, both through guiding and managing the process of insurance plan approval and also prescription renewal and medicament delivery or collection.

The application may also provide holistic care by providing access to learning material including instructional articles and videos and motivational articles and challenges, and by providing a connection to other connected devices, such as activity trackers and heart rate monitors to continuously update and learn about user's behavior. The application may also gather and combine information from other programs such as environmental monitoring apps or weather forecasts.

The application can be used to manage a broad range of diseases which are treated by a range of different medicaments. Although some of the specific embodiments below are described in relation to the treatment of atopic dermatitis and/or asthma using a single medicament, the application is not so limited. Various other immunology indications may also be managed by the application, where one or more of these immunology indications may be treated by using a single medicament approved for use in treating the one or more immunology indications. Such a single medicament can include, for example, different dosages containing the same API, different volumes or concentrations of the same API, or different formulations containing the same API. In some embodiments, a single medicament can include an anti IL-4R mAb (e.g., Dupilumab). Further, a broad range of immunological conditions may be managed by the application. The user may be prescribed one or more drugs and the application provides personalized support for users depending on their particular drug prescription(s) and health profile. For example, the application may be configured to provide support for a number of diseases caused by Type 2 inflammation. In the context of dermatology this may include Atopic Dermatitis, Prurigo Nodularis, Bullous Pemphigoid, an Urticaria (e.g., chronic spontaneous urticaria, or cold inducible urticaria), Hand and Foot Disease, or Pruritis, each of which may be treated by injections of Dupixent® or other injectable drugs, but also by some oral and topical medications. In the context of respiratory diseases, this may include Asthma, Nasal Polyps, Sinusitis (e.g., chronic sinusitis with or without nasal polyps), Allergic Bronchopulmonary Aspergillosis (ABPA), or Allergic Fungal Rhinosinusitis (AFRS) (which may be treated by injections of Dupixent® or other injectable drugs but also by some oral medications and Chronic Obstructive Pulmonary Disease (COPD, which may be treated by injections of Dupixent® or other injectable drugs. In the context of Gastroenterology this may include Inflammatory Bowel Disease (IBD) and Eosinophilic Esophagitis (EoE), Eosinophilic Gastroenteritis (EGE), or Ulcerative Colitis, which may be treated by injections of Dupixent® or other injectable drugs but also by some oral medications.

The medicament may be administered by injection. As used herein, the term injection or self-injection is intended to encompass intra-venous injection, intra-muscular injection, infusion, or any needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

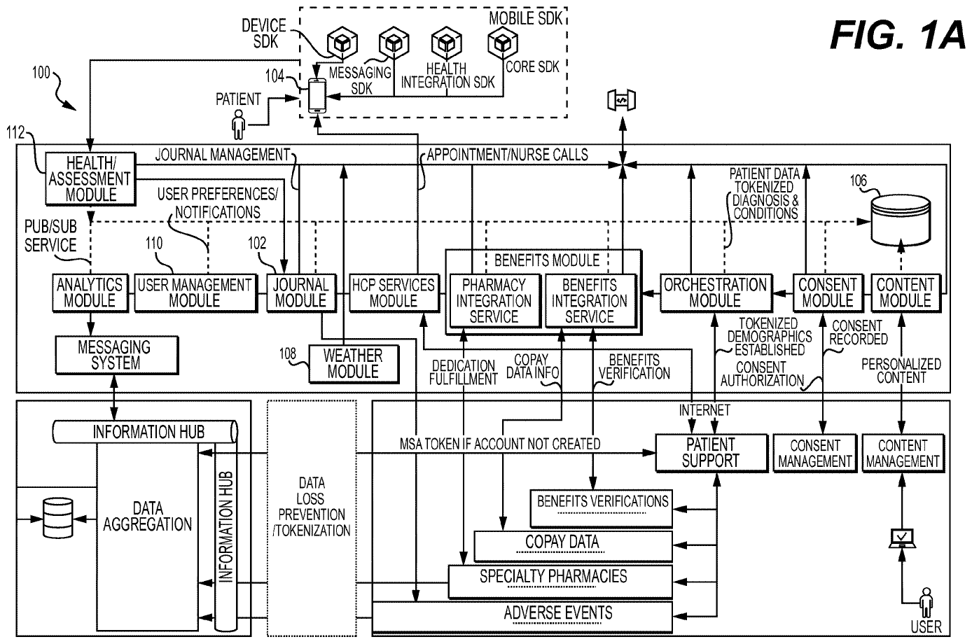

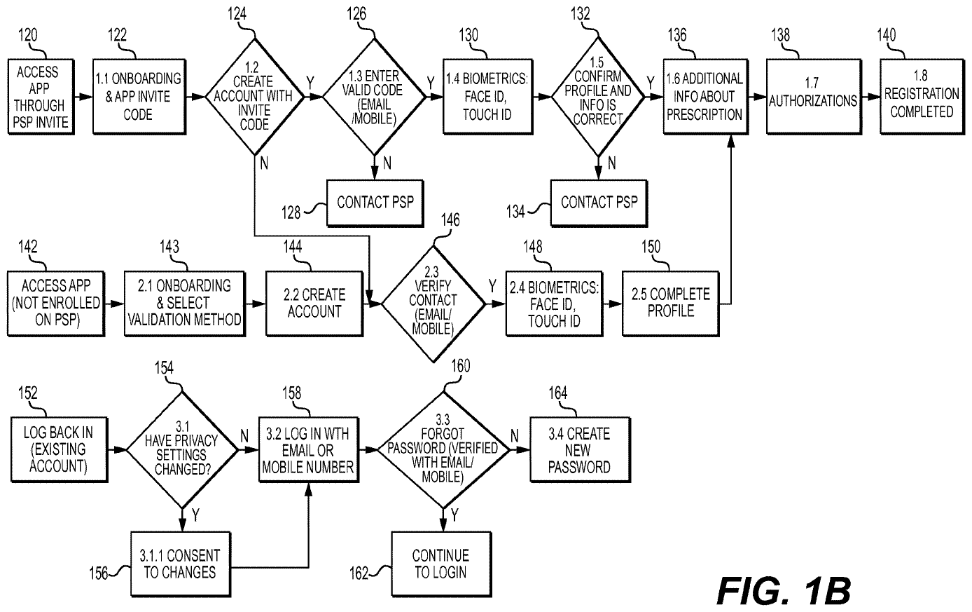

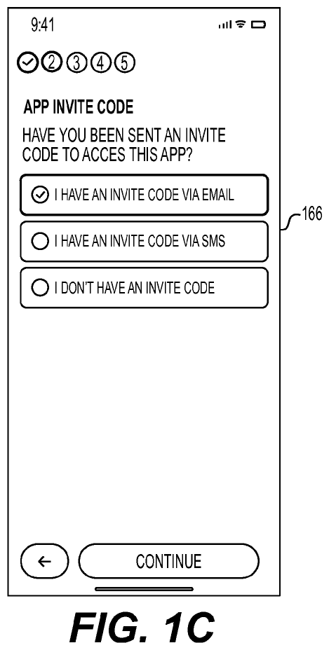

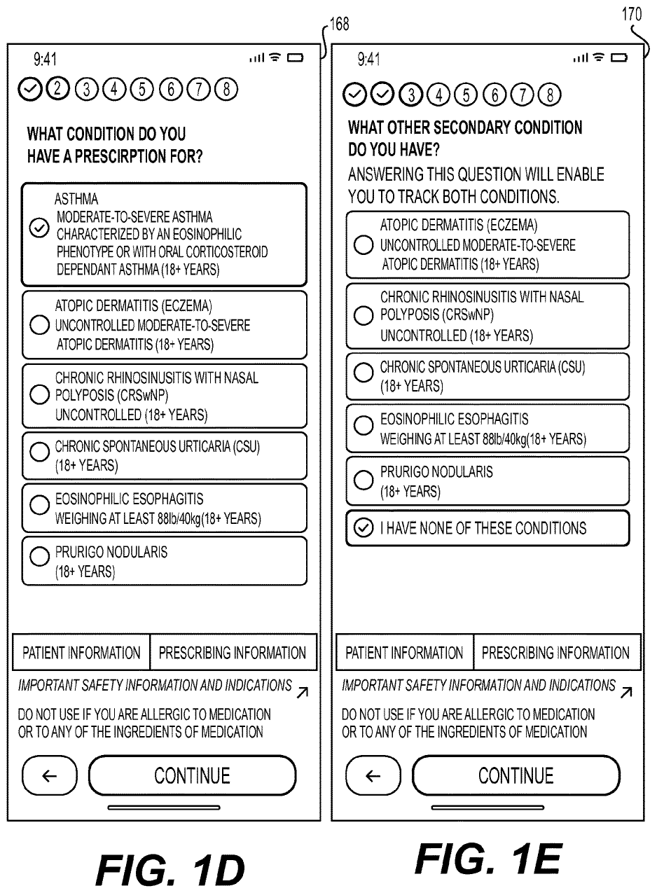

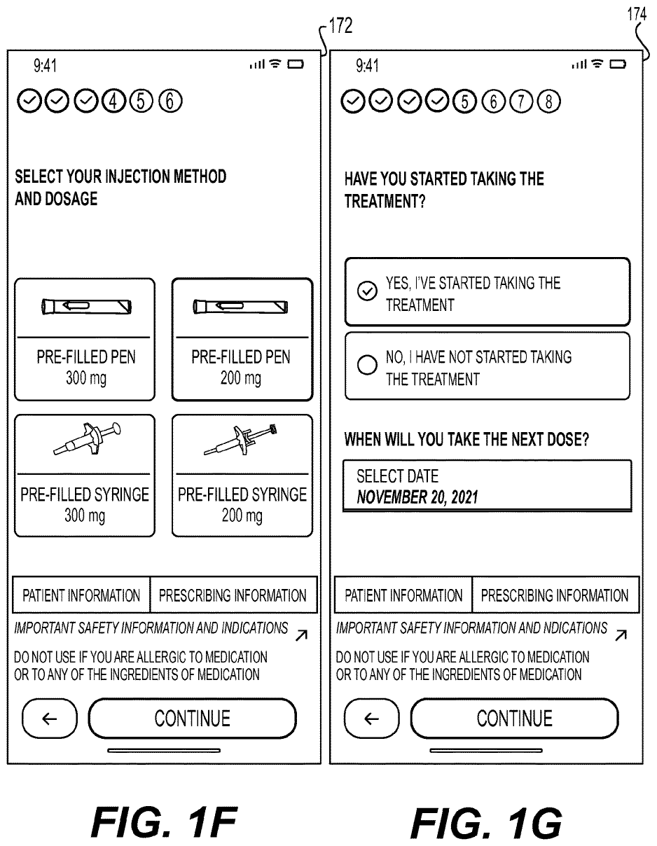

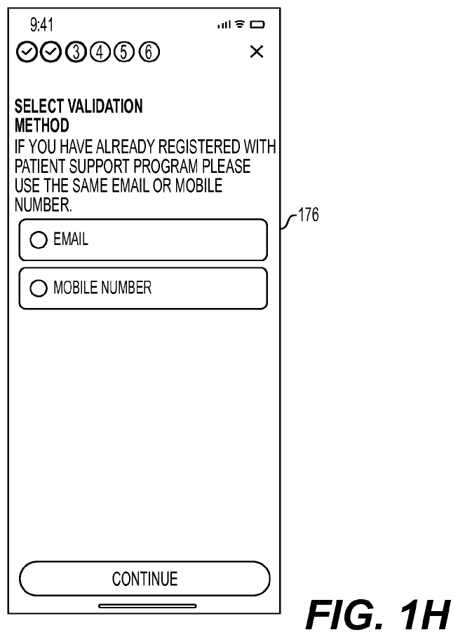

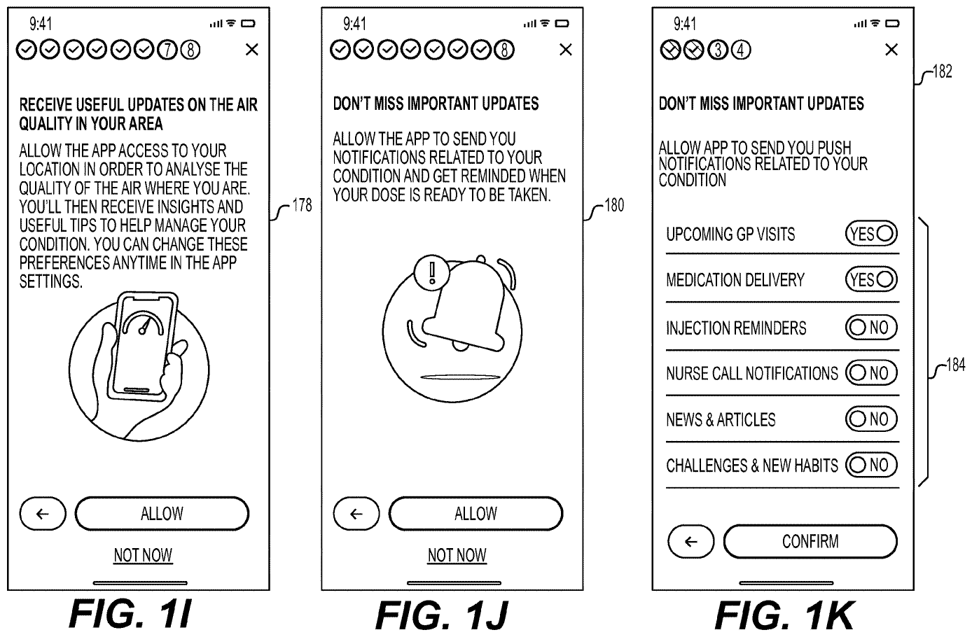

Referring to FIG. 1a, schematic overview of an overall architecture 100 for a disease management and medication regimen management system is shown. The system architecture 100 illustrates a number of functional modules and the data links between these. The system architecture 100 comprises a user device 104 running an application with a number of functional modules, illustrated in the central box. The architecture 100 also illustrates how the application run by the user device 104 can interact with other service providers to enhance the information which can be provided to the user through the application. For instance, by communicating with insurance/benefit provider systems and pharmacies.

One of these functional modules is a journal module 102. The journal module 102 is configured to allow a user to maintain a journal relating to one or more medical conditions of the user. The journal may comprise a plurality of journal entries that detail episodes of the one or more medical conditions, such as symptoms, user specific health data, contextual information or the like. The journal may be used to monitor the one or more medical conditions for patterns, and to identify potential triggers for episodes of the one or more medical conditions. The journal may further maintain a record of the adherence of the user to a treatment regime, such a medicament regime. For instance, the journal entry can include a record logging a dose of medicament (e.g. a scheduled dose) that has been taken by the user, such as an injection log. The record can form a dose record including a variety of information associated with the dose administered by the user. The record can be stored as part of the journal entry or independently from the journal entry. The journal module 102 may communicate with an external service called adverse events. The journal module may transmit patient information associated with episodes of the one or more medical conditions to the adverse events service.

The functional module may further comprise a memory module 106. The memory module 106 is configured to store journal entries made by the user via the journal module 102 for later retrieval and/or analysis. The memory module 106 may, in some embodiments, be linked to the cloud, and store journal entries remotely.

In some embodiments, the functional modules may further comprise a weather module 108. The weather module 108 is configured to determine and/or record weather conditions at the location of the user. The user location can be determined, for example, by a GPS capability of the user device. The weather module 108 may, for example, access an online weather forecasting system to obtain current weather conditions, previous weather conditions and/or predicted future weather conditions. The weather module 108 may provide weather conditions to the journal module 102 for inclusion in journal entries. As well as weather conditions such as general weather condition, temperature, humidity, air pressure and UV index, the weather module may additionally obtain air quality information such as pollen count, pollution index, NO₂ count and/or an air quality index.

Another of the functional modules is a user management module 110. The user management module 110 is responsible for obtaining user preferences and for generating notifications for output on the user device 104. Necessary data for the notification scheme may be stored on the memory module 106. The user management module 110 may also update the information held in the memory module 106 as the application is used. The user management module 110 may define and help implement a notification scheme which determines when a notification should be generated and how it should be output on the user device 104. The notification scheme may have three main notification types; (i) a silent notification; (ii) a batched summary notification; and (iii) a push notification.

In some embodiments, the silent notification may be pushed into the application silently and viewable only within the application. The user may be presented with the silent notification within the application, for example on an application home screen. In some embodiments, the silent notifications are surfaced to the user when certain requirements are met and do not require any action on user's part. The batched summary notification may relate to several different reminders and other notifications. The user can interact with the batched summary notification to expand and show the individual notifications. The batch summary notification may indicate the number of notifications which are present for the user to review. The push notification may relate to a single notification and is pushed to the lock screen or home screen of the user device 104. In some embodiments, the push notifications are time-sensitive and actionable items that require the user's immediate attention. They are reserved for actions related to the user's treatment routine or as a follow up to actions that can impact the user's access to the treatment/drug.

Notifications can escalate, i.e. move up the notification types, depending on the user's action (or lack thereof). For example:
a. When the user is 5 days away from their next medicament dose, their next dosing reminder will show up as a silent notification in the application, for example underneath the carousel on the App homepage.
b. In the days leading up to the dosing, the application will push batch notifications to the user to either review instructional videos, book a call with a nurse, or review the instructions for self-administration.
c. On the day of the dosing and an hour prior to the scheduled time, the application will push a notification to the user to take their dose. If the user's medication is kept in the fridge, the notification will instruct the user to take their medicament out of the fridge. Interacting with this notification will trigger a warm-up timer, set to their medication dose.

Another of the functional modules is a Health/Assessment Module 112. The Health/Assessment Module 112 is responsible for onboarding the user and obtaining details regarding the user's medical conditions and medicaments being used to treat these. The Health/Assessment Module 112 may also gather information about the user's prescriptions and manage delivery of medicaments.

Another of the functional modules is a Health Care Professional (HCP) Services Module. The HCP Services Module is responsible for facilitating contact with a nurse, doctor or other health care professional, including scheduling reminders relating to appointments and calls, prompting the user to create reports or otherwise prepare for an upcoming appointment or call, initiating voice or video calls, providing follow-up notifications after calls or appointments and initiating a journal entry after calls or appointments. The HCP Services Module interacts with an internet-based Patient Support Program. Health care professionals may also have access to certain aspects and information held by the Patient Support Program, in order to facilitate contact with the user and monitor the user's treatment and/or disease progression.

Another of these functional modules is a Consent Module (also referred to as a consent management module) which, in conjunction with other aspects of the system, is responsible for managing consents and permissions for accessing patient specific information, such as health related data for the user. The Memory Module 106 may be configured to store user ID and role information as well as consent templates for use by the Consent Module. The Memory Module 106 may also store consent and permission information input by the user via the consent Module, including any time limits set on the consents and permissions.

The Consent Module may communicate with an external service called Consent Management service. The consent and permission information generated by the Consent Module may be stored or copied to the Consent Management service. The Consent Management service may also be responsible for sending the patient specific information to third parties when the associated consents indicate that this is permitted.

Another of the functions modules is a Content Module. The content module is responsible for receiving and recording personalised content and/or preferences set by the user. The content module may communicate with an external service called Content Management. The content management service responsible for providing patient specific content or settings from an external user, such as a healthcare professional. The content module 124 may interact with the memory module 106 to store the personalised content and/or settings.

Another of the functional modules is a Benefits Module which, in conjunction with other aspects of the system, is responsible for managing patient access to health care. The Benefits Module may include a pharmacy integration service and/or a benefits integration service. The information generated by the benefits module may be responsible for sending patient specific information to third parties to indicate patient access to health care services. The benefits module may interact with external services, such as benefit provider systems and pharmacy services.

Another of the functional modules is an Analytics Module. The analytics module may be responsible for analysing the patient information associated with the status and/or treatment of the user's medical conditions.

Another of the functional modules is a Messaging System. The Messaging System may communicate with the analytics module. For instance, if the analytics module identifies an episode or a trigger to an episode of the one or more medical conditioners of the user, the messaging system outputs a message communicating this result. The messaging system may communicate with an external service called an Information Hub. The information hub may store the data received from the messaging system. The information hub may also interact with other service providers to enhance the information which can be stored in respect of the user through the application. For instance, by communicating with the patient support program, insurance/benefit provider systems and pharmacies, adverse events services, and so on.

Referring to FIG. 1b, a flowchart is shown illustrating the overall process of user account creation and login.

At step 120 the user accesses the application through a patient support portal (PSP) invite. In this scenario, the user is already enrolled with the patient support portal and receives an application invite code, either via email or SMS. At step 122, the application displays an onboarding page, for example by displaying the screenshot of FIG. 1c. At step 124, the application determines whether the user has selected to create an account using an invite code. For example, the user provides an input indicating that they have received an invite code (YES at step 124). In this case, the flow proceeds to step 126 in which the application displays a page asking the user to enter their invite code. If the user does not enter a valid invite code (NO at step 126), the application may offer the user the option to contact a representative of the patient support portal at step 128.

When the user enters a valid invite code (YES at step 126), at step 130 the application provides the option to enable biometric login control, such as fingerprint recognition or facial recognition. Biometric login is however not mandatory. At step 132, the application displays a screen summarizing information received by the application for the PSP, which may for example include the user's name, email, date of birth and gender. In this step, the application also presents options for the user to confirm that the information is correct or to indicate that it is incorrect. If the indicates that some aspect of the profile information is incorrect (NO at step 132), the application may offer the user the option to contact a representative of the patient support portal at step 134.

When the user confirms that the information is correct (YES at step 132), at step 136 the application displays one or more screens asking the user to enter information about their prescription. In some embodiments this may be a single screen with several GUI elements in which the user can enter information. In another example, the application may show a screen similar to that in FIG. 1d allowing the user to select from a list of conditions which they have a prescription for. After receiving a user input selecting a first condition, the application may display a screen similar to that in FIG. 1e asking the user if they have any secondary conditions and allowing the user to select from a list of conditions which they have. After receiving a user input specifying condition and any secondary conditions, the application may display a screen similar to that in FIG. 1f allowing the user to select their prescribed injection method and dosage. After receiving a user input selecting their prescribed injection method and dosage the application may display a screen similar to that in FIG. 1g allowing the user to specify whether they have started taking the medication or not. If the user indicates that they have started taking the medication, the screen may display further GUI elements for the user to indicate when their next scheduled dose is. In this manner a current medicament dose schedule can be set in the application during account creation.

After collecting the additional prescription information, at step 138 the application gathers any necessary permissions to send the user notifications and to monitor the user device location. At this time, the application may also gather any necessary permissions to hold or process the user's personal data in accordance with laws in the local jurisdiction. At step 140, the application displays a registration completed page.

In an alternative account creation process, at step 142 the user accesses the application without an invitation. In this scenario, the user is not enrolled with the patient support portal. At step 143, the application displays an onboarding page, for example by displaying the screenshot of FIG. 1c. In step 144, a user input is received indicating that the user does not have an invite code. In response, at step 146, the application asks the user to select a verification method, which may be one of email or mobile phone number. For example, the application may display a screen similar to that in FIG. 1h. The process may also arrive at step 146 if the user was invited to access the app with an invite code from the PSP, but in step 124 selected that they did not have a code (e.g. because they had lost or not received the code)

After successfully completing the validation process by email or mobile phone number (YES at step 146), the application provides the option to enable biometric login control, such as fingerprint recognition or facial recognition, in the same way as in step 130. At step 150 the application displays one or more screens asking the user to complete their profile information, by entering personal information, such as the user's name, email, date of birth and gender. After entering this information, the process continues to step 136, as described above.

FIG. 1b also illustrates a process flow for logging back into the application. At step 152, the user attempts to log back into the application at some time after account creation. In step 154 the application checks whether there has been any update to the privacy policy/settings on the application. If there have been changes (YES at step 154), then at step 156 application displays a page providing a link to any updated policies and asking the user to consent to the storing and processing of their data in line with the new policies. When the user consents to the new privacy policies/settings or if there has been no update of these, then the process continues to step 158, in which the application beings a log in process. At step 160 the application checks if the user has indicated that they have forgotten their password. If the user has not forgotten their password (NO at step 160), then at step 162, the process continues to the log in page. If the user has indicated that they have forgotten their password (YES at step 160), then the application begins a password reset process in step 164.

When the application is first installed and run by the user, an application registration and profile creation routine is performed. This routine may include, among other steps, the user creating an account by inputting an email and password. Verification codes may be sent to the user's email which must be entered into the application to complete the account creation. The application may also be configured for biometric login and may ask the user if they wish to use stored biometric login information (such as fingerprint(s) or facial recognition) as the primary means for logging into the application. After creating an account, the user may be guided through the steps of creating a profile including their personal and contact details, the medical conditions that they have been diagnosed with and the dates of these diagnoses.

FIGS. 1c to 1h show screenshots illustrating an account creation process for the application. FIG. 1c shows an application invitation screen 166. This screen may be displayed on the first occasion that the user accesses the application, and asks the user whether they have received an invite code and by what channel. Where a user is already enrolled with a patient support portal (PSP) relating to a prescribed medicament or to a condition which the user has, then they may generate an invite code via the PSP (or a HealthCare Professional or the PSP operator may initiate generation of the invite code). The application invitation screen 166 has three selectable options to indicate either (i) that the user has received an invite code via email, (ii) that the user has received an invite code via SMS or (iii) that the user has not received an invite code. If the user selects either option (i) or (ii) then they are prompted to enter the code. The application may communicate with the PSP to validate the entered code. Any personal information held by the PSP may be communicated to the application, so that the user does not need to enter this information manually. If the user selects option (iii) then the application may prompt the user to enter an email address or mobile phone number through which the validation process can be conducted.

After the user has been validated, a condition selection screen 168 is displayed, as shown in FIG. 1d. The condition selection screen 168 may comprise a list of conditions which are treated by a first medicament and the user may select which of the conditions they have a prescription for. The list of conditions may include Asthma, Atopic Dermatitis (Eczema), Chronic Rhinosinusitis with Nasal Polyposis (CRSwNP), Chronic Spontaneous Urticaria (CSU), Eosinophilic Esophagitis and Prurigo Nodularis. Not all of these conditions may be included in the list and this list is not exhaustive. In some embodiments, the user may select more than one condition on the condition selection screen 168. After selection to continue, the application displays a secondary condition selection screen 170, as shown in FIG. 1e. The secondary condition selection screen 170 may contain the same list of conditions as the condition selection screen 168, minus any that the user has already selected. The secondary condition selection screen 170 may also contain an option for the user to indicate that they have no further conditions.

After indicating the conditions they have, the application displays an injection method and dosage selection screen 172, as shown in FIG. 1f. The medicament prescribed to the user may come in a range of different dosages and delivery methods and these may be indicated with selectable graphical icons, images and/or wording. The four delivery methods illustrated in FIG. 1f are merely exemplary. A subset of these, or different delivery devices may be displayed depending on the condition or conditions selected by the user. After selecting which method and dosage they have been prescribed, the application displays treatment start screen 174, as shown in FIG. 1g. The treatment start screen 174 comprises options for the user to indicate whether they have started taking their treatment/medication already or have not yet started taking their medication. If the user selects the option to indicate that they have started taking their medication already, then a further GUI element may appear or become accessible in which the user can input the date of their next scheduled dose. In this manner, a current dosing schedule can be set in the application during the account creation process. If the user selects the option to indicate that they have not started taking their medication, then a further GUI element may appear or become accessible in which the user can input the date on which they plan to take their first dose. Each of the prescription information gathering screens shown in FIGS. 1d-1g may display links to important or useful information such as safety information relating to the medicament.

After entering the information discussed above, the application displays a summary screen (not shown) with information on the user's dose, method of administration, date of next scheduled dose and dose frequency. This page may offer the option for the user to contact a support service if any of the information appears to be incorrect.

After displaying the summary screen, the application may display a first authorization screen 178, as shown in FIG. 1i. The first authorization screen 178 relates to receiving air quality updates via the application. Air quality may have a direct effect on the user's symptoms and so providing this information via the application can help the user to manage their condition. If the user selects to allow the application to provide air quality information, then the application may request access to the location of the user device, which the user may approve (either for the current session, or constantly) or deny. Air quality and location represent one example of information which may be customized according to the health conditions being managed. Other kinds of information which may be informative or valuable for the user to be made aware of may also be communicated, such as weather information or traffic data. The application may determine that certain information may be of value to the user based on the specific health conditions they are managing and that access to the user's location data or some other protected data stored on the user device or relating to the user's health condition(s) is needed to customize the information being presented. The application can then present a user interface asking for authorization to access the protected data so that the information can be customized.

After receiving the user input relating to air quality information, the application may display a second authorization screen 180, as shown in FIG. 1j. The second authorization screen 180 relates to receiving notifications from the application relating to the user's condition and treatment. If the user selects to allow the application to provide notifications, then the application may display notification consent screen 182, as shown in FIG. 1k. The notification consent screen 182 may show a number of user selectable options 184 for different types of notifications. The user selectable options 184 may be toggle button switchable between “yes” and “no” and may comprise topics such as “Upcoming GP visits”, “Medication delivery”, “Injection reminders”, “Nurse call notification”, “News & articles” and “Challenge & new habits”. Selecting the yes toggle for any of these options 184 allows the application to send the user push notification relating to the associated topic. The push notification may be displayed on the locked screen of the user device 104. After selecting the notification options, the user may be presented with various policy statements, such as a privacy policy statement. The user's permission to collect and store their person data in particular ways may also be obtained at this point.

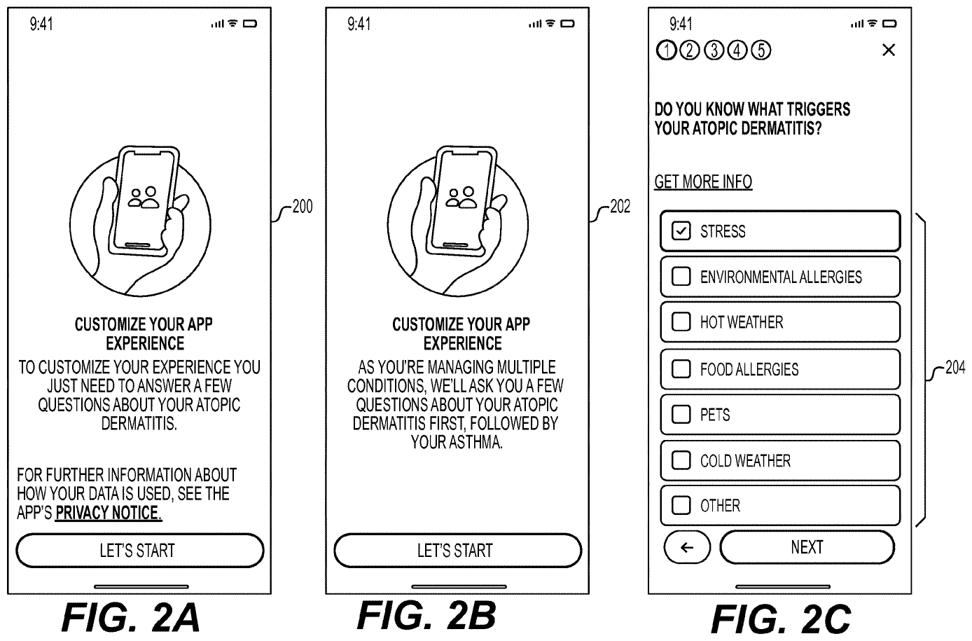

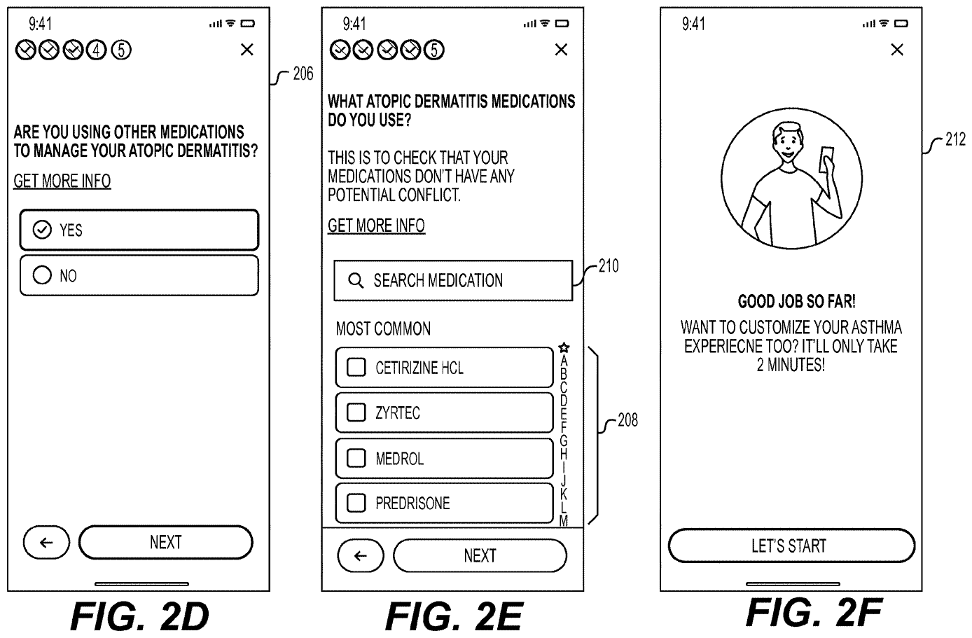

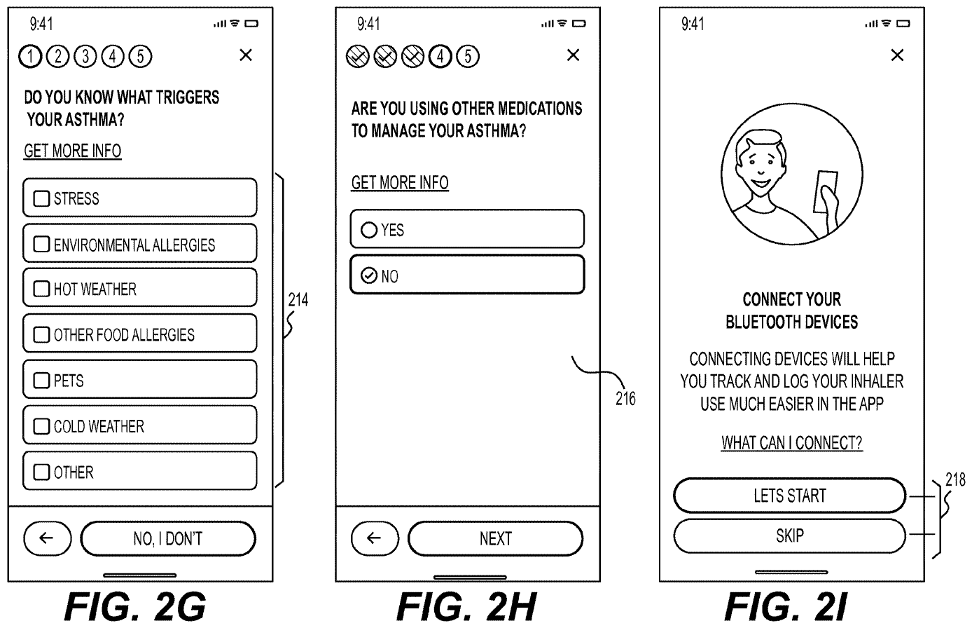

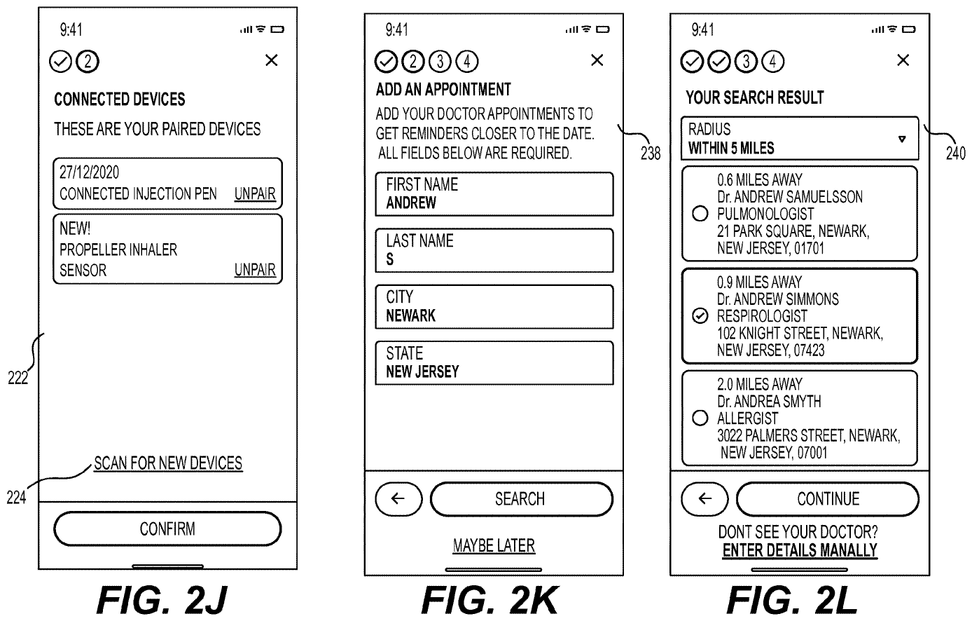

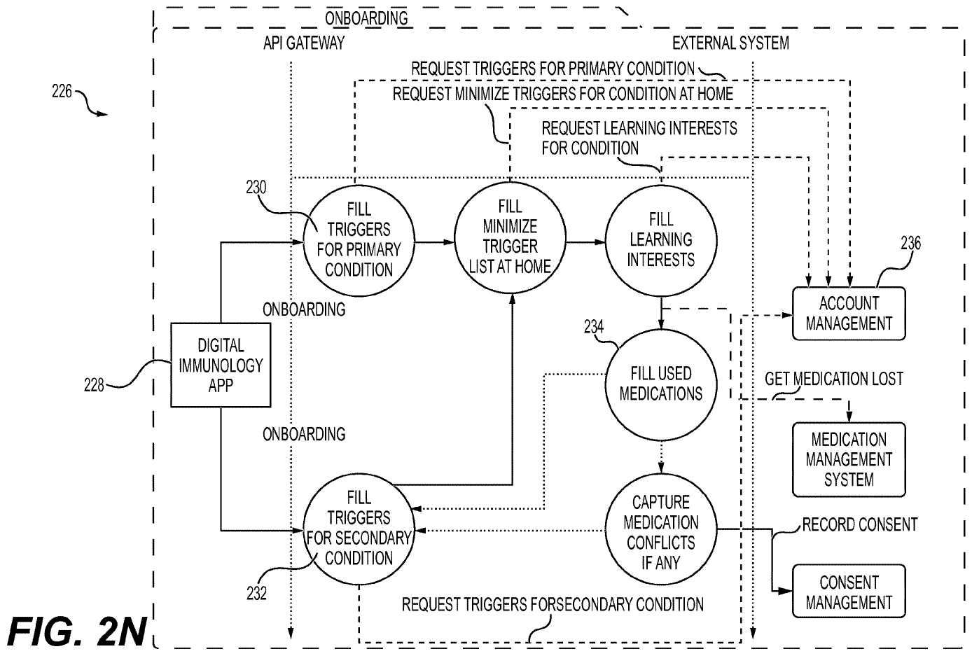

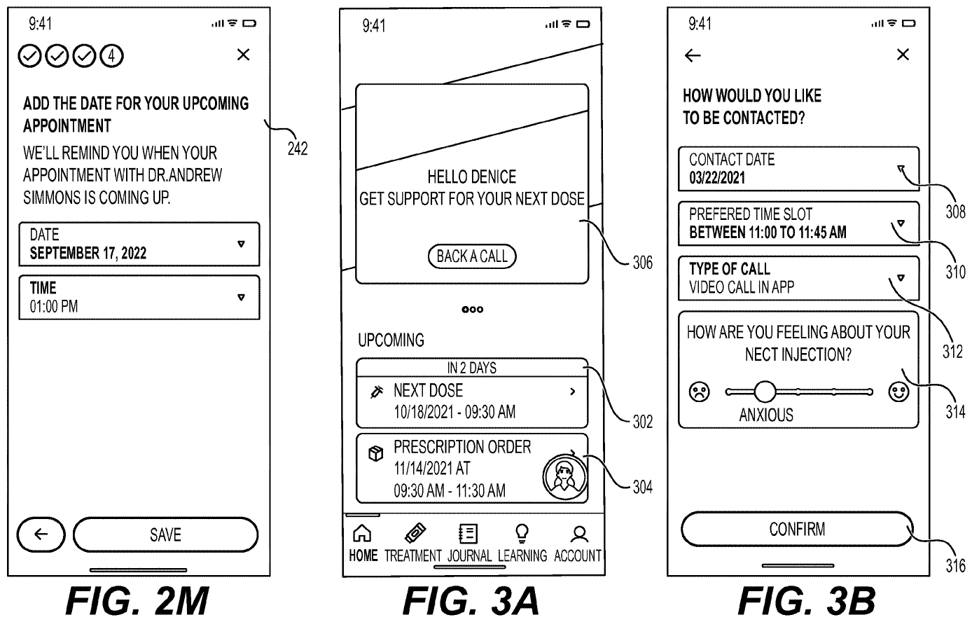

Referring now to FIGS. 2a to 2h, screenshots illustrating an application customization process are shown.

After completing their profile, the application may allow the user to perform a customization process. The customization process may be accessed from a notification on the home screen of the application or may be begun automatically after profile creation. FIG. 2a shows a first initial screen 200 of the customization process. The first initial screen 200 may be shown when the user has indicated during account creation that they are managing one health condition. In the example shown in FIG. 2a, the user has selected Atopic Dermatitis. FIG. 2b shows a second initial screen 202 of the customization process. The application is configured to manage multiple health conditions of the user, and the second initial screen 202 of the customization process may be shown instead of the first initial screen 200 when the user has indicated during account creation that they are managing multiple health conditions. For clarity, although the application is capable of handling more than one health condition, the user may select just one of the health conditions that the application is configured to handle, and the application will in that event provide support for only the selected health condition. In the example shown in FIG. 2b, the second initial screen 202 informs the user that the application will ask for information about both Atopic Dermatitis and Asthma. As previously described, the application may be programmed to manage a range of immunological or other medical conditions being treated by a particular medicament or group of medicaments.

FIG. 2c shows the first screen of the customization process relating to Atopic Dermatitis. The first screen has a list of trigger conditions 204 and asks the user to select those which they believe are triggers for their Atopic Dermatitis. The user may select as many of the trigger conditions 204 as they wish. There may also be an “other” option and a box for entry of free text associated with this option. The customization process may then ask the user what steps they currently take to minimize trigger conditions, by presenting a list of options for user selection and/or allowing free text entry. The customization process may then ask the user what topics they are interested in learning about, again by presenting a list of options for user selection and/or allowing free text entry. The user selection of these topics may inform what is displayed to them on the “Learning” tab screen of the application (see FIG. 14).

FIG. 2d shows an additional medicaments question screen 206 in which the user is asked if they are using other medicaments to manage their Atopic Dermatitis, giving “yes” and “no” selectable options. If the user selects “yes”, then the additional medicaments selection screen of FIG. 2e is displayed. The additional medicaments selection screen contains a list of additional medicaments 208, with each entry in the list having an associated check box. The list may display a number of common medicaments at the top, followed by a list of medicaments in alphabetical order. The additional medicaments selection screen also has a search box 210 with which the user can search the list of additional medicaments 208.

If the user were only managing Atopic Dermatitis, then the customization process would then proceed to the connected devices prompt screen, as shown in FIG. 2i. When the user is managing multiple health conditions, after the users selects any additional medicaments they are using for the first health condition, the customization process displays a second medical condition prompt screen 212, as shown in FIG. 2f. The second medical condition prompt screen 212 displays a notification prompting the user to customize the application in relation to a second medical condition, in this embodiment, Asthma. If the user chooses to end the customization process at this point, then a notification prompting the user to customize the application in relation to a second medical condition may be displayed prominently on the home screen of the application.

When the user continues the customization process, first screen of the customization process relating to Asthma is displayed, as shown in FIG. 2g. This screen has a list of trigger conditions 214 and asks the user to select those which they believe are triggers for their Asthma. The user may select as many of the trigger conditions 214 as they wish. There may also be an “other” option and a box for entry of free text associated with this option. FIG. 2h shows an additional medicaments question screen 216 in which the user is asked if they are using other medicaments to manage their Asthma, giving “yes” and “no” selectable options. If the user selects “yes”, then an additional medicaments selection screen similar to that of FIG. 2e and with similar functionality, is displayed. Additional screens (not shown) may be displayed during the customization process for Asthma, asking the user to specify which preventer inhaler they user and which reliever inhaler they use. The application may then ask the user if they are taking any medications other than the prescribed medicament managed by the application and their inhalers to manage their asthma.

Similar to the process described above, if the user has indicated that they have been prescribed the medicament to manage Chronic Rhinosinusitis with Nasal Polyposis (CRSwNP), Chronic Spontaneous Urticaria (CSU), Eosinophilic Esophagitis and/or Prurigo Nodularis, then for each health condition selected, an separate customization process may occur in which the application displays a screen (similar to that of FIGS. 2c and 2g) asking the user if they know what triggers the condition, a screen (similar to that of FIGS. 2d and 2h) asking the user if they are using any other medication to treat the condition and a screen (similar to that of FIG. 2e) providing a list of other medicaments for the user to select. The list of trigger conditions and list of other medicaments may be different for each health condition.

After selecting any additional medicaments being used to manage each of the user's health conditions, the customization process shows a connected devices prompt screen, as shown in FIG. 2i. The connected devices prompt screen may comprise options 218 to add connected devices, or to skip this part of the customization process. The connected devices prompt screen may also comprise a selectable link 220 to information about which connected devices are compatible with the application. Selecting the link may navigate to such information within the application, or in some embodiments may be a URL link configured to open an internet browser.

When the users selects the option 218 to add a connected device, a connected devices management screen 222 is shown, as illustrated in FIG. 2j. This screen lists any connected devices capable of wireless communication and associated with the user. The user can use this screen to pair a new device, such as an injector or an inhaler, with the application for managing the user's medical condition or treatment, or remove any unwanted devices. The connected devices management screen 222 comprises a user selected option 224 to scan for new devices, for example using a Bluetooth interface of the user device 104 or a NFC protocol. Alternatively, the user's injector or inhaler may have a scannable code, such as a QR code (or similar barcode) printed on a label affixed to the device, or otherwise printed or etched onto the device. Upon selecting the option to connect a new device, the application may offer the option to open the camera app of the user device. The camera of the user device can then be used to capture the scannable code, such as the QR code. The type and dosage of the injector or inhaler can then be checked against the information entered during the account creation process. Alternatively, this step may replace the manual entry of the injection device information during account creation. Scanning a QR code on an injection device allows the application to check the expiry date of the medicament within the injection device. If the medicament is expired, this can be communicated to the user via a warning in the application.

The QR codes may serve a further purpose of directing users towards the application or to automatically begin the account creation process. After being prescribed the medicament and receiving an injection device, the user can scan the QR code with their device. If the application is not installed on the user device, then scanning the QR code may open an app store on the user device and navigate to the application store page, where the application can be downloaded and installed. Alternatively, a page may be displayed notifying the user that the application is available and providing a link to open the app store. If the application is already installed on the user device, then scanning the QR code may cause the application to automatically launch and begin the account creation and customization process described herein. If the user is an existing user of the application and they scan a new injection device, then the application may automatically launch and ask the user to confirm that they have received a new injection device or batch of injection devices. This may cause the refill reminder in the application to be reset.

As an alternative to providing QR codes (or similar barcode) on a label on the injection devices, the QR codes (or similar barcode) may be provided on a box containing the injection devices.

The actions resulting from scanning the label on the box may be the same as described above. The application may continue the customization process by displaying an appointment entry screen 238, as shown in FIG. 2k. The appointment entry screen 238 asks the user to enter details of a scheduled doctor's appointment, so that reminders can be generated. The screen 238 may have a number of free text entry boxed for entering the doctor's first and last names, city and state/region. The screen 238 may have a selectable soft button to commence a search for the doctor and an option to skip the appointment entry process. When the user enters information into at least one of the boxes and presses the search button, a search is conducted and a search result screen 240 displayed, as shown in FIG. 2l. The search results screen 240 may comprise a number of search results and a filter for setting a distance (radius) of the search. The search results screen 240 allows the user to select one of the search results and continue. The search results screen 240 also has an option to enter the doctor's details manually. Once the doctor has been selected or the details entered manually, the process continues to an appointment details entry screen 242, as shown in FIG. 2m. This screen may have data entry boxes for the date and time of the appointment. After saving the appointment details, the customization process is complete.

Referring now to FIG. 2n, a data flow diagram 226 is shown, illustrating the requests for user data input in the oval boxes in the center of the diagram and the movement of data (labelled arrows) between the application and external systems and services. The “Digital Immunology App” 228 is the user facing end of the application described above. An Application Programming Interface gateway is programmed into the application for exchanging data with functional modules, such as the Health/Assessment Module 112 described with reference to FIG. 1. The user interacts with the Digital Immunology App 228 to begin an onboarding process (also referred to as customization process), as described above with reference to FIGS. 2a and 2b. At step 230, the application asks the user to “Fill triggers for a primary condition”. In the embodiment described above, the application shows the screen of FIG. 2c, asking the user about trigger conditions for their Atopic Dermatitis (the primary condition). The process then proceeds to ask the user what steps they currently take to minimize trigger conditions for the primary condition, by presenting a list of options for user selection and/or allowing free text entry. The process then asks the user what topics they are interested in learning about in relation to their primary condition, again by presenting a list of options for user selection and/or allowing free text entry. The information entered by the user in each of these steps is communicated to an external account management service 236, which can include a data sever which stores user specific health information.

At step 234, the application asks the user to “Fill used medications”. In the embodiment described above, the application shows the screens of FIGS. 2d and 2e, asking the user if they are taking other medicaments to manage the primary condition and what these medicaments are. As indicated in the data flow diagram 226, the list of medicaments may be provided by an external “Medication management system”. This allow the list of medicaments presented in FIG. 2e to be kept up-to-date, without the need to provide an update to the application.

As indicated in the data flow diagram 226, an additional step of “Capturing medication conflicts if any” may be performed. The details of medication conflict may also be stored in the external Medication management system. If any medication conflicts are found, a warning may be displayed to the user as part of the customization process. The warning may advise the user to contact their health care provider to discuss the potential conflict.

After collecting the information about what other medicaments the user is taking, the process continues to step 232, in which the application asks the user to “Fill triggers for a secondary condition”. In the embodiment described above, the application shows the screen of FIG. 2g, asking the user about trigger conditions for their Asthma (the secondary condition). The process then cycles back and proceeds to ask the user what steps they currently take to minimize trigger conditions for the secondary condition, by presenting a list of options for user selection and/or allowing free text entry. The process then asks the user what topics they are interested in learning about in relation to their secondary condition, again by presenting a list of options for user selection and/or allowing free text entry. The information entered by the user in each of these steps is again communicated to the external account management service 236.

At step 234, the application again asks the user to “Fill used medications” in relation to the secondary condition. In the embodiment described above, the application shows the screens of FIG. 2h and a screen similar to FIG. 2e but adapted for Asthma, asking the user if they are taking other medicaments to manage the secondary condition and what these medicaments are. As indicated in the data flow diagram 226, the list of medicaments may be provided by an external “Medication management system”. This allow the list of medicaments presented in FIG. 2e to be kept up-to-date, without the need to provide an update to the application.

At the end of the process, the applicant may ask for the user's consent to store the information they have entered as part of the customization process. A dedicated external “consent management” system may handle this aspect of the process and may store the evidence of the consent requests shown to the user and the user's responses.

FIG. 3a shows a dose support screen of the application. The dose support screen provides information regarding scheduled medication doses of a user. The dose support screen may comprise one or more reminders/notifications of upcoming events relation to one or more medications of the user. For example, the dose support screen may show one or more reminders/notifications for a scheduled medication dose 302. The reminder for a scheduled medication dose 302 may comprise a medication identity and a time (e.g. date and time) that the medication dose should be taken. Alternatively or additionally, the dose support screen may show one or more reminders/notifications for a scheduled medication delivery 304. The reminder for a scheduled medication dose 304 may comprise a medication identity and a time (e.g. date and time) that a delivery of the medication is scheduled for.

The dose support screen may further comprise an interface for a call-scheduling feature 306. The call-scheduling feature 306 facilitates booking a call between the user and a healthcare professional, e.g. a nurse, for guiding the user through the administration of their medication, e.g. how to prepare and administer an injection. The user may select a GUI element, e.g. a button, to initiate the call-scheduling feature 306. If the call scheduling feature is initiated, the process proceeds to FIG. 3b.

As shown in FIG. 3b, following initiation of the call-scheduling feature 306, the user inputs their contact preferences. The contact preferences provide details about the user's preferred methods of being contacted. The contact preferences may, for example, comprise a preferred contact date 308, a preferred contact time (or time window) 310, and a preferred contact method 312 (e.g., in-app video call, phone call, external app video call etc.). In some embodiments, the user may also provide input 314 indicating how they feel about their next medication dose. Once the details have been provided, the user may activate a confirmation GUI element 316 to save the preferences and transmit them to an analytics platform. As an alternative, to the screen shown in FIG. 3b, the user may request a support call immediately. In response, the application may check whether the support service is currently operational (e.g., within operation hours). If this is the case, the application may ask the user what their call is about, so that they can be directed to an appropriate person for support. The application may then open the phone application, (or another communication program) of the user device and dial the appropriate number, so that the support call can begin immediately.

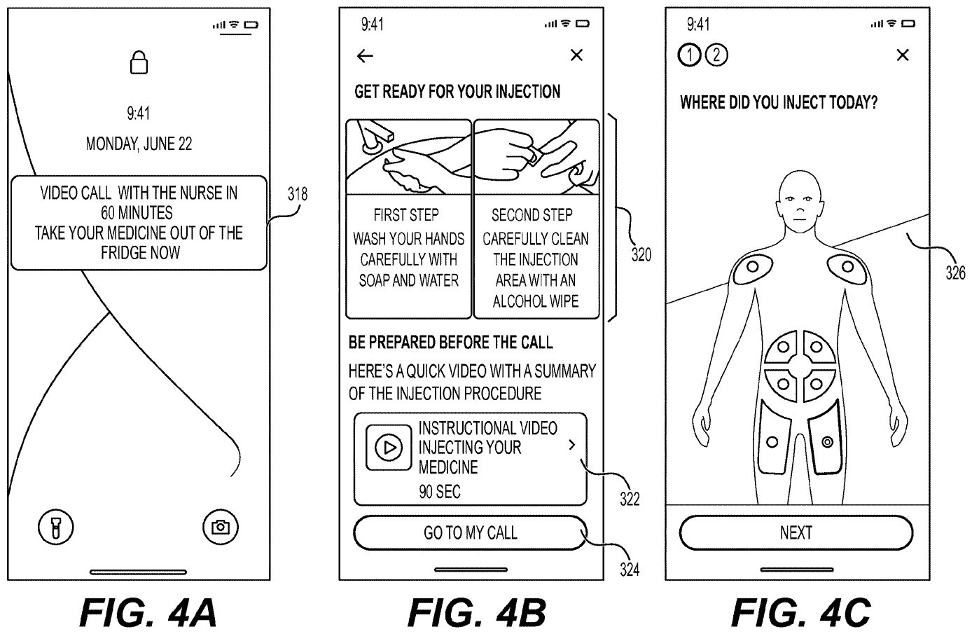

FIG. 4a shows an example of a notification 318 for an upcoming call with a healthcare professional. The notification is pushed to a lock screen of the user device. The notification provides a reminder of the time remaining until the call and the identity of the person the call will be with (e.g. “Video call with the nurse in 60 minutes”). The notification may further provide instructions for actions the user should perform in advance of the call, such as instructions to take the medication out of storage a predefined time before the call, e.g. “Take your dose out of the fridge now”. The user may select the notification to open the application on the user device.

Upon selecting the notification, or opening the app and navigating to the call feature, the user may be presented with an instruction screen, such as the one shown in FIG. 4b. The instruction screen provides instructions 320 for preparing a medication dose in advance of a scheduled call with a healthcare professional. The instructions may comprise photographic instructions and/or text instructions. In some implementations, video instructions 322 may also be provided. When the call time has arrived, the user may select the “Go to call” 324 element to proceed to the call, during which the user will administer their medication under the remote supervision of the healthcare professional.

Following the call, the application may display a notification asking the user if they took their medication while on the call. If the user indicates that they did not take their medication, they may be presented with an option to set a reminder for taking the medication at a later time. If the user indicates that they did take their medication, the user may be requested to input additional data relating to the administration of the medication. For example, as shown in FIG. 4c, the user may select an injection location 326 from a set of predefined locations to indicate where the user administered the injection.

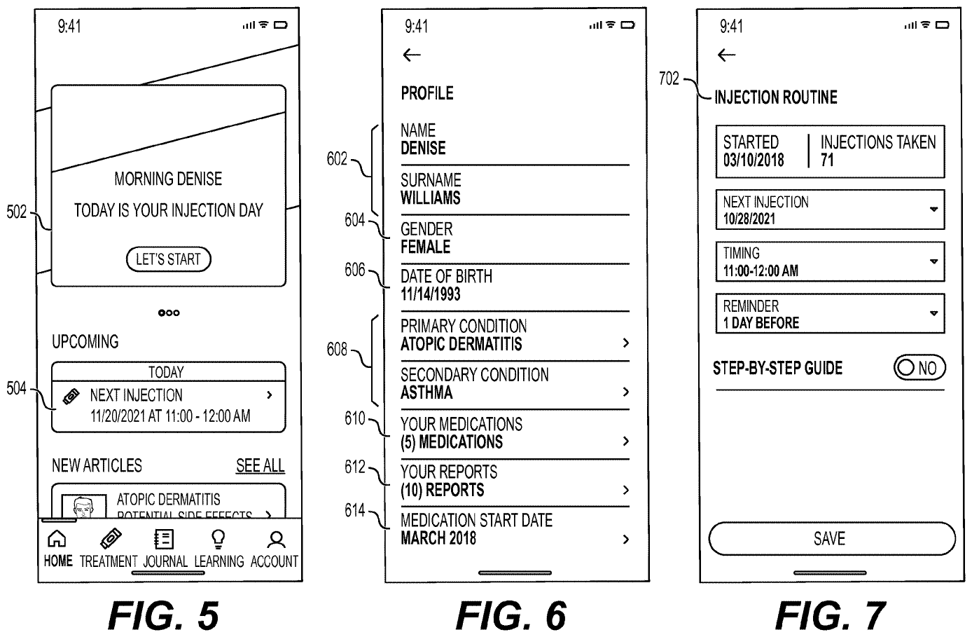

FIG. 5 shows an example of a home screen of the application. The home screen shows a prominent reminder 502 that today is the day the user should take their medication. A calendar notification 504, providing the time window in which the medication should be taken, is also provided. Selecting either the reminder 502 or the calendar notification 504 may begin a medicament dose support process in which the application shows a step-by-step dosing guide, e.g. a step-by-step injection guide. The medicament dose support process may also include reminding the user to take their medicament out of the fridge and providing a warm-up time period countdown timer. In addition, the application may display a push notification on the lock screen of the user device 104 at the scheduled time of the next dose. Selection of this push notification may also begin the medicament dose support process by the application once the user device has been unlocked.

FIG. 6 shows an example of a user profile in the application. The user profile may comprise: a user identity 602, e.g. the name of the user; a user gender 604; a user date of birth 606; a plurality of medical conditions of the user 608; one or more medications taken by the user 610; one or more reports regarding the medical condition 612; and/or a medication start date 614. As can be seen, the profile indicates that the application is assisting the user with managing a primary condition (Atopic Dermatitis) and a secondary condition (Asthma). Further conditions may also be managed by the application, for example immunological conditions as described above. The profile also indicates that the user may be using more than one medicament to manage the conditions. In the embodiment of FIG. 6, five medicaments are indicated (610). The user can select an entry in the profile to view more details about the entry, such as a list of the individual medicaments. The profile information is viewable and editable by the user at a later time. The user's treatment details, including which health conditions they are managing, which other medication they are taking, the things which trigger their symptoms and which injection method and dosage they are using can also be viewed and edited at a later time.

FIG. 7 shows an injection routine control screen 702. The injection routine control screen 702 allows the user to specify at what time of day they usually perform their injection, so that they can be reminded at an appropriate time. The user may also use this screen to specify how long before the scheduled injection time they wish to be reminded about the injection, for example 1 day before. There is also a selectable option for whether to provide a step-by-step guide as part of the injection reminder. This option may be toggled to ‘yes’ by default until the user specifies otherwise. The option to skip the step-by-step guide may not be available, or may not be available until the application determines that the user has sufficient experience, for example once they have completed a certain number of doses. Although this screen is described in terms of an injection reminder, the application is also able to assists the user in managing other forms of medication administration, such as oral and topical medicaments.

The timing of the notifications, in particular the push notification and batch notification may initially be defined by the application. However, the application may also dynamically adjust the timing of the notifications as the user engages with the application. The application can learn about particular user behavior and can track when the user is most active and when they are most likely to open the application. The application can then adjust the timing of notifications accordingly for maximum impact and maximum user engagement.

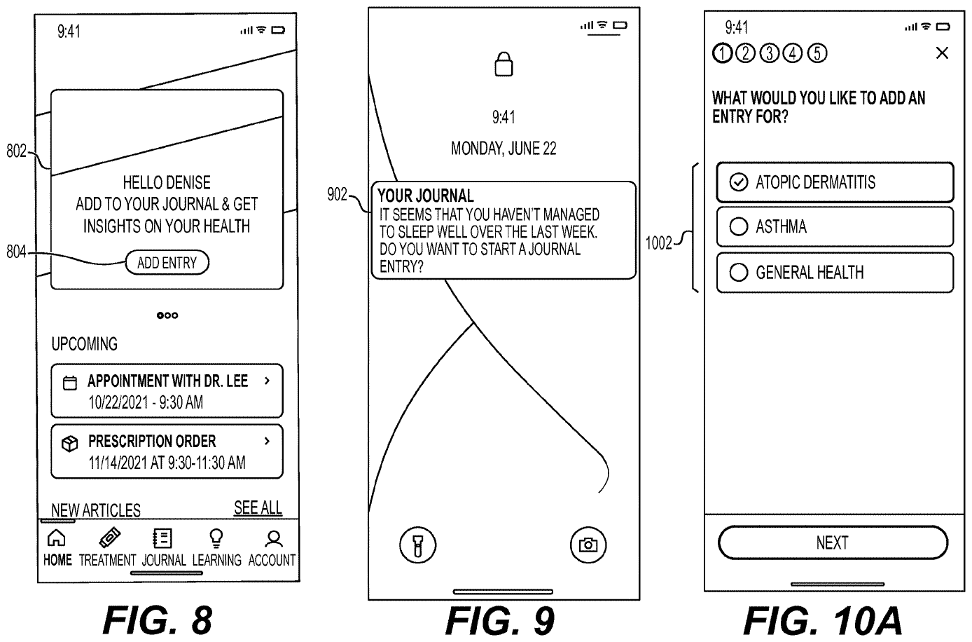

FIG. 8 shows an example of a graphical user interface for the application comprising in-application notification 802 prompting the user to make a journal entry. The notification may be triggered when the user starts up the application if no daily journal entry has been made that day, and prompts the user to start their daily journal entry. Alternatively or additionally, the notification may be triggered by the user entering the application via a lock screen notification, as described below in relation to FIG. 9. The notification may comprise a GUI element, e.g. a button 804, which may be selected by the user to open the journal module at the entry for the current day. In the example shown, the GUI button for accessing the journal is marked “Add Entry”.

FIG. 9 shows a push notification 902 to a locked screen of a user device prompting the user to make a journal entry based on detected environmental conditions or user specific health information.

The application monitors environmental conditions of the user environment, and triggers a notification 902 if the one or more environmental threshold conditions are met. The notification 902 may provide a prompt to the user to make a journal entry and, in some embodiments, provide a warning about the environmental condition that triggered the notification. The application may monitor the environmental conditions using one or more sensors present on the user device, such as a thermometer, barometer or hygrometer. Alternatively or additionally, the application may monitor the environmental conditions using environmental information from one or more other services, for example a weather application on the device or on the internet.

The occurrence or parameters of this notification may be further determined, in part, by whether the user has previously selected an environment-related trigger (e.g., hot weather, cold weather, and/or environmental allergies) for the user's primary or secondary condition (e.g., in steps 204, 214, 230 and/or 232). For example, a “high temperature” notification may be presented to the user more urgently if the user has indicated that “hot weather” triggers the user's asthma than if the user has not.

The application may alternatively or additionally monitor user specific health information, and trigger a notification if the one or more health condition thresholds are met. The notification may provide a prompt to the user to make a journal entry and, in some embodiments, provide a warning about the health information that triggered the notification. The user specific health information may be input by the user into the device, for example through a GUI of the device. Alternatively or additionally, the user specific health information may be collected by one or more sensors of the device, for example, a user pulse may be measured by an optical sensor on the device. Alternatively or additionally, the user specific health information may be collected from one or more external devices. For example, the application may have access to data collected by a wearable device of the user, such as a smart watch, and/or a medical device used by the user to monitor a health condition, such as a pulse oximeter, or a device for monitoring the user's breathing such as a peak flow meter or spirometer, or the like.

The occurrence or parameters of this notification may be further determined, in part, by whether the user has previously selected a health-related trigger (e.g., stress) for the user's primary or secondary condition (e.g., in steps 204, 214, 230 and/or 232). For example, a “high pulse” notification may be presented to the user more urgently if the user has indicated that “stress” triggers the user's asthma than if the user has not.

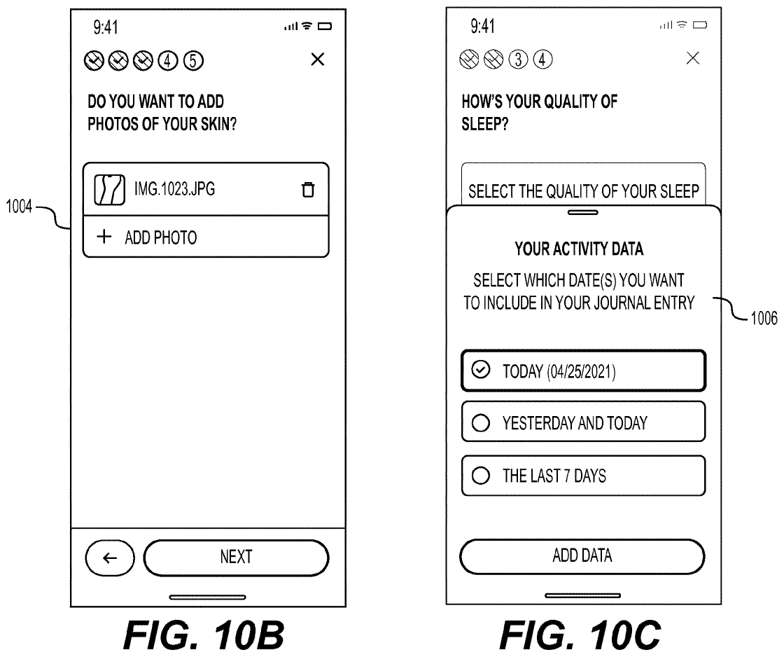

FIG. 10a shows a health condition entry screen of the application. The user selects one or more health conditions 1002 that the journal entry relates to from a health condition selection page. Selection of the one or more health conditions 1002 may be performed by clicking on an associated tick-box, as shown in the figure. Alternatively or additionally, the one or more medical conditions 1002 may be selected from one or more drop-down lists and/or input manually. In the example shown the medical conditions for the journal entry comprise atopic dermatitis and asthma, as well as a general “overall health” condition. In the example shown the user has selected atopic dermatitis as the relevant medical condition. Once the user has selected the one or more relevant medical conditions 1002 for the journal entry, the user may select a “next” button to proceed to the next part of the journal entry.

The process of making a journal entry may comprise the user providing location and time details relating to an episode of the medical condition and answering one or more queries regarding the health condition. The queries may be answered by selecting a symbolic score (e.g. a face) or a numerical score (e.g. a number between zero and five) from a set of potential scores. The selection may be performed using a slider, though other methods of inputting the scores may alternatively be used, e.g. a drop-down box, a text input or the like. In some embodiments, one or more of the queries may be in the form of a “yes/no” or “true/false” statement. The queries may relate to aspects of the health condition (e.g. for atopic dermatitis, “What is your level of itchiness?”, “How is the level of pain?” etc.) or to the general condition of the user with respect to the health condition (e.g. “How are you feeling now?”, “How did you sleep last night?”). If the user is making a journal entry for Asthma, the queries will be different, e.g. “What level is your shortness of breath?” etc. or to the general condition of the user with respect to the health condition e.g. “How are you feeling now?”. Having provided answers to the queries, the journal entry process may display a model of the human body, with which the use can indicate particular body locations at which the health condition, e.g. atopic dermatitis, is occurring. The model may be a rotatable model, allowing the user to select locations on the front, back and sides of the body. Such information may only be relevant to certain health conditions, and as such, this journal page may not be present in all situations (such as for Asthma). The user may indicate one or more locations at which the health condition is occurring/an episode of the health condition is occurring. If the user has previously indicated that the health condition is occurring at one or more body locations, the user may indicate which locations are getting worse.

Two further steps may be part of the journal entry process. FIG. 10b shows a photograph entry screen 1004. For certain health conditions, for example Atopic Dermatitis, the journal entry process may ask the user if they wish to add photographs of their skin, in order to better show and track the severity of the condition. The photograph entry screen 1004 may show a thumbnail of any photographs which have already been uploaded as part of the journal entry and contain a button for adding a photograph. Selection of the button may present the user with the option to open a camera application on the user device or a gallery application on the user device, in order to capture or select a photograph to add. A separate free-text entry screen may also be presented to the user as part of the journal entry process.

As part of the journal entry process, the user may be asked about the quality of their sleep, to which they can input a numerical score, symbolic score, or free-text entry. The user may also be presented with the option to add sleep quality data. Selection this option may cause the application to display an activity tracker data selection screen 1006, as shown in FIG. 10c. In this embodiment, the activity tracker is a “Google Fit”, however the device may be any suitable activity monitoring device. The device may have been paired with the application during the customization process (see FIG. 2j). If no activity tracker device was previously paired, selection of the option to add sleep quality data may open a connected device pairing screen and being a search for nearby devices. On the activity tracker data selection screen 1006, the user may be presented with a list of time periods to associate with the journal entry, which may comprise for example “Today”, “Yesterday and Today”, “The last 7 days”, or any other suitably defined time period. Associating activity data with the journal entry can allow the application to derive further insights into the user's health condition and to correlate activity with symptoms and present this information to the user at a later time.

Having answered the queries relating to a first health condition (and optionally indicated one or more locations on the body model), the user may be asked about a second health condition, if it was selected by the user using the input in FIG. 10a. In this manner, a journal entry relating to multiple health conditions being treated by a medicament can be made in a single process.

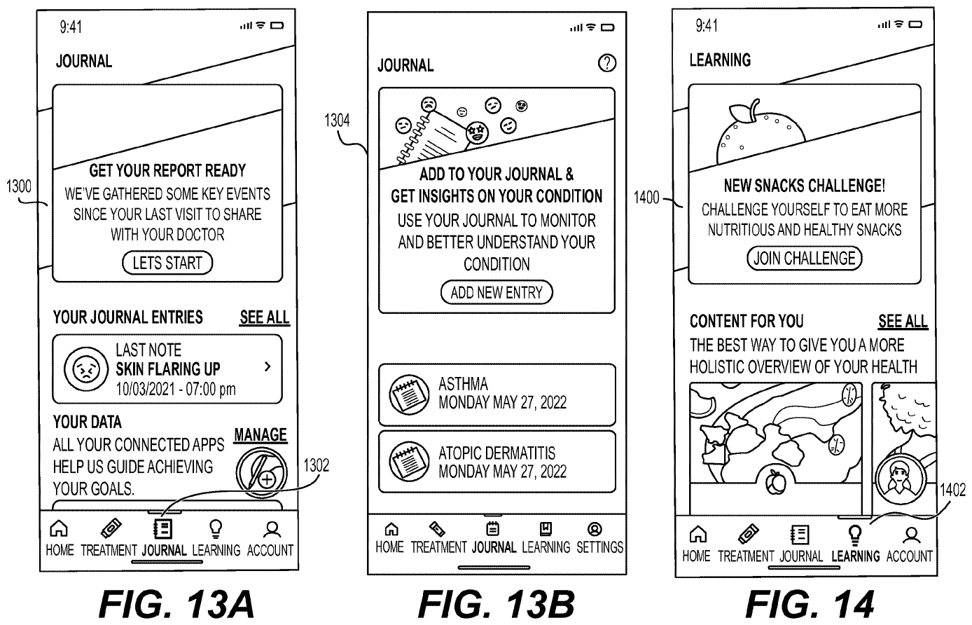

After completing a journal entry, the user may be presented with a journal reminder screen in which they can set a day and time at which they wish to be reminded to make a further journal entry. The application stores a record of the user's journal entries. The user can access and view this record from the journal tab (see FIGS. 13a and 13b).

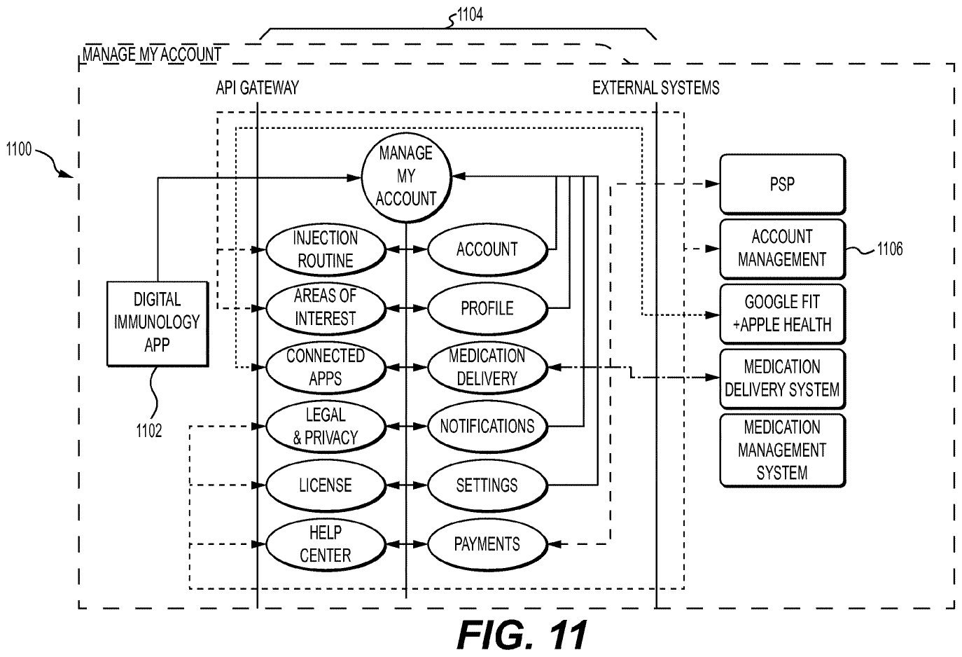

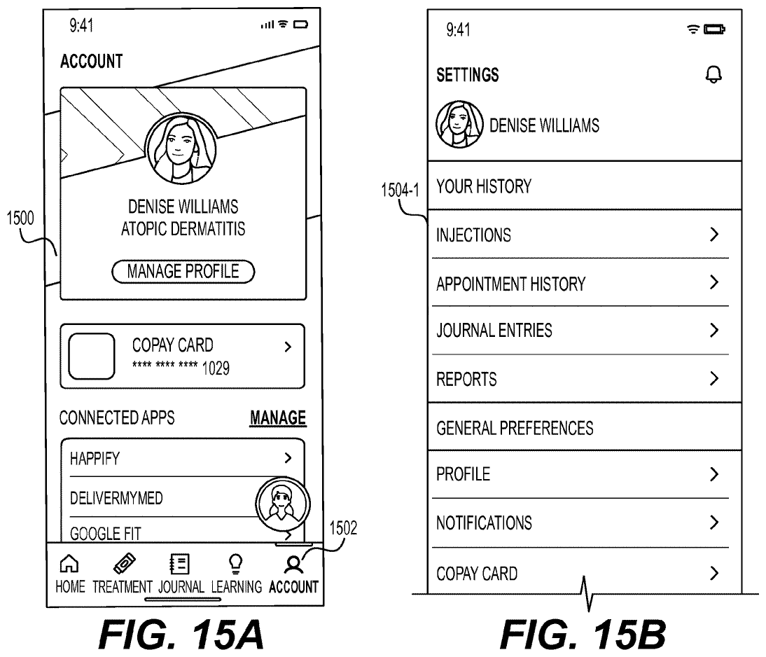

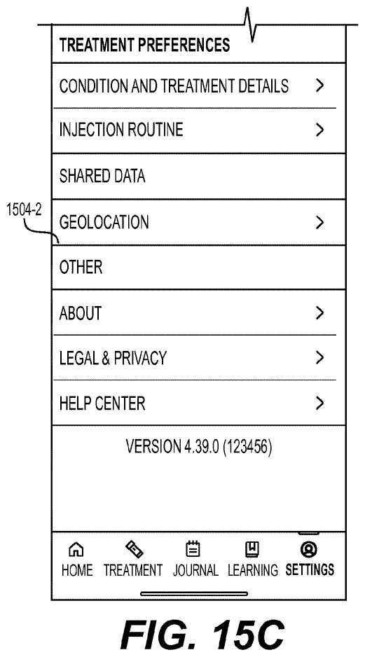

In describing the features of the application above, reference has been made to various external systems and device with which the application communicates and exchanges data. FIG. 11 is a data flow diagram 1100 illustrating categories of data in the oval boxes in the central section 1104 of the diagram and the movement of data (labelled arrows) between the application and external systems and services. The “Digital Immunology App” 1102 is the user facing end of the application described above. An Application Programming Interface gateway is programmed into the application for exchanging data with functional modules, such as the User Management Module 110 and Health/Assessment Module 112 described with reference to FIG. 1. The user interacts with the Digital Immunology App 1102 to manage their account, which they may do by selecting the “Account” tab in the application (see for example FIG. 15).

Within the account management section of the application, various categories of data can be viewed and edited as represented by the oval boxes in the central section 1104 of the diagram. These may include, but are not limited to injection routine, account, areas of interest, profile, connected apps, medication delivery, legal and privacy, notifications, license, settings, health center and payments. The information entered by the user in respect of some of the categories is communicated to an external account management service 1106, which can include a data sever which stores user specific health information. Information relating to payments may be exchanged with a patient support portal (PSP). Information relating to connected apps and/or devices is exchanged directly with those apps and devices for example “Google fit” or “Apple health”. Information relating to medication delivery may be exchanged with one or more medication delivery systems, such as a connected injection device or a connected inhaler device (see FIG. 2j). The Medication management system may store, and provide when queried, a list of medicaments and information about medicament conflict.

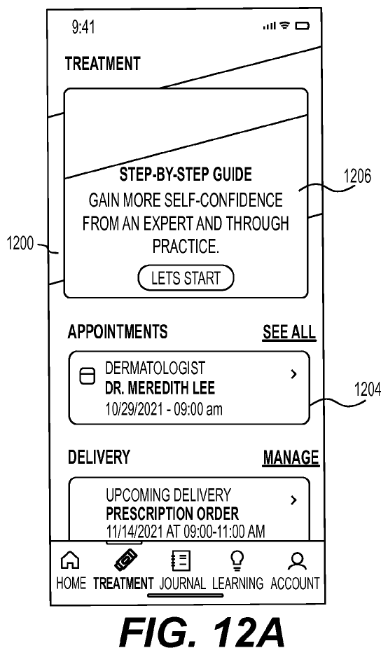

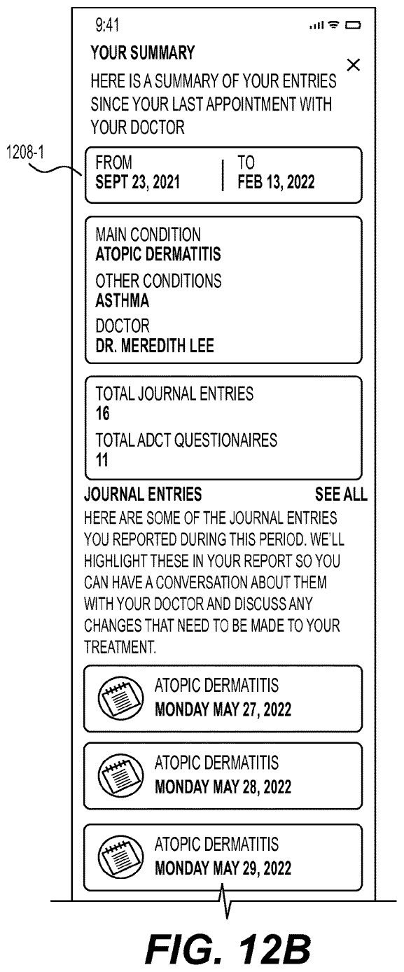

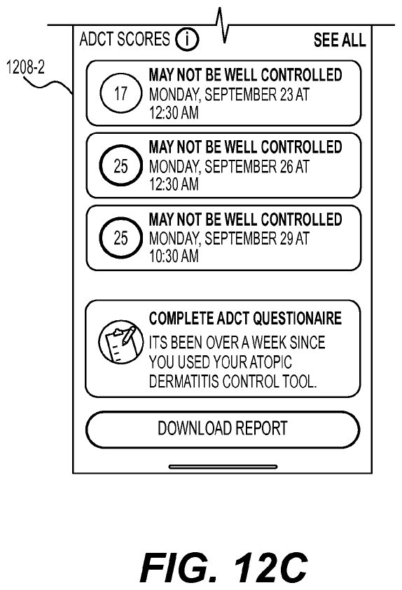

The application also assists the user in preparing for appointments relating to their treatment, for example doctor's appointments. FIG. 12a shows a treatments tab screen 1200, which is shown within the application when the treatments tab 1202 is selected. The treatments tab screen 1200 comprises a notification 1204 reminding the user of an upcoming doctor's appointment and giving some details for the appointment. The treatments tab screen 1200 also comprises a suggested action graphic 1206, which in this embodiment is to access a step-by-step injection guide. Selecting the button within the suggested action graphic 1206 may navigate to information within the application, such as video or graphical instructions or may open a web browser on the user device and navigate to the information. In this manner, the user is better able to prepare for appointments relating to treatment of their condition. Other types of suggested action graphic may appear in this section of the treatments tab, for example a notification to get a repot ready for an upcoming doctors' appointment. The type of notification shown may depend on which of a scheduled dose or scheduled appointment is soonest.

The treatment tab screen 1200 may also provide access to prescription and delivery information to allow the user to track when a new delivery of their medicament is due. The treatment tab screen 1200 may also provide access to a dosing history, for example an injection history. This allows the user to view a complete list of all of the injection events they have inputted into or have otherwise been recorded in the application.

Selecting the notification 1204 may open a new screen giving more details about the appointment, for example that it relates to a monthly check and may also provide a selectable option to prepare a report in advance of the appointment. FIGS. 12b and 12c show a report screen (1208-1, 1208-2) which can be generated by the application. The report may comprise a summary of the user's journal entries and questionnaires completed, so as to detail the progression of their health conditions. The summary may relate to a certain time period. By default, the time period may begin on the day after the user's previous doctor's appointment and end on the current day. The time period may be adjustable by the user. Under the summary of the journal entries, the report screen 1208-1 may show the most recent journal entries individually and indicate which condition or conditions they relate to. For example, the three most recent journal entries may be shown. The user may select any individual journal entry to view the full entry. The report screen 1208-1 may also provide a “see all” selection, allowing the user to view a complete list of their journal entries. Under the journal entries, the report screen 1208-2 may show a list of the user's most recent questionnaire scores. In the example depicted these are Atopic Dermatitis Control Tool (ADCT) scores. A summary of the result of each questionnaire may also be given, e.g. “Well controlled” or “May not be well controlled” as well as the date and time on which the questionnaire was completed. For example, the three most recently completed questionnaires may be shown. An option to complete a further questionnaire may be provided. Finally, a selectable GUI element to download the report is provided. The report can then be shared with the user's doctor or other healthcare professional.

Both the journal entries and the questionnaires are disease specific. Therefore, if the user has more than one health condition that they are managing with the application, then each journal entry may relate to one or more health conditions and the results of different questionnaires may be shown. For example, if the user has Asthma, then the questionnaire may relate to the Asthma Control Tool (ACT). If the user has Prurigo Nodularis, then the questionnaire may relate to the Prurigo Nodularis Control Tool (PCT). If the user has Chronic Spontaneous Urticaria, then the questionnaire may relate to the Urticaria Control Tool (UCT). Some diseases, such as Chronic Rhinosinusitis with Nasal Polyposis (CRSwNP), or Eosinophilic Esophagitis may not have a specific questionnaire associated with them, and in this case the summary report may omit the questionnaire section shown in FIG. 12c.

Any correlations which have been noted between the user's reported symptoms and other information in the journal entries (such as environmental conditions, sleep or user activity data) may also form part of the report. In this manner the application is able to gather and present therapeutically useful information to the user and their healthcare professionals at an appropriate time. The user may generate, view and comment on reports at other times. Generating the report may also involve the user answering a short questionnaire about their current symptoms for each of the health conditions they are managing with the application. The application may provide an option to send the report in electronic form directly to the doctor after it has been produced.

FIG. 13a shows an exemplary journal tab screen 1300 which is displayed when the journal tab 1302 is selected within the application. The journal tab screen 1300 may comprise a notification about the most recent journal entry made by the user. The user may select this notification to view the journal entry and/or view all journal entries. The notification may also have a separate selectable element to view a list of all journal entries. The application may also automatically generate graphs and charts from the numerical information contained in the journal entries to graphically depict the user's treatment, symptoms and/or disease progression. These graphs and charts may additionally or alternatively be generated on demand. The journal tab screen 1300 may also comprise a suggested action graphic related to journaling, such as to prepare a report to share with their healthcare professional. The journal tab screen 1300 may also comprise a selectable option to add a connected device so that the data collected as part of the journaling can be enhanced with data from external sources.

FIG. 13b shows an alternative journal tab screen 1304. The alternative journal tab screen 1304 comprises a suggested action graphic related to journaling, in this instance to add a new journal entry. The alternative journal tab screen 1304 comprises a list of the most recent journal entries. These can be selected to view the full entry or a complete list of past journal entries.

FIG. 14 shows an exemplary learning tab screen 1400 which is displayed when the learning tab 1402 is selected with in the application. The learning tab screen 1400 may comprise personalized content for the user which is selected based on the one or more medical conditions which they are managing using the application and in accordance with the profile preferences input in FIG. 2a and the user's indicated topics of interest set during the customization process. The content may comprise articles and/or videos. The learning tab screen 1400 may also comprise a suggested action graphic, for example a suggested to join a challenge. If selected, the application may present information about a way in which the user can adjust their behavior for improved health, for example dietary information. The application may also assist the user in tracking their progress through the challenge. An alternative suggested action may be to view a step-by-step injection guide. The learning tab screen 1400 may also contain links to view safety information and indications for the medicament.

FIG. 15a shows an exemplary account tab screen 1500 which is displayed when the account tab 1502 is selected with in the application. The categories of information which can be viewed and managed in the account tab screen 1500 have already been described with reference to FIG. 11. The account tab screen 1500 may comprise a link to open a profile management page, for example, the page shown in FIG. 6. The user's currently connected external devices may be listed on the account tab screen 1500 as well as links to payment and insurance information.

FIGS. 15b and 15c show a setting tab screen (1504-1, 1504-2), which may be presented as an alternative to the account tab screen 1500 of FIG. 15a. The settings tab screen has a number of sections for viewing and editing information. The first section is a history section, via which the user can view their past injections, appointments, journal entries and generated reports. The second section is a profile section, via which the user can view and edit their personal information. For example, selection “profile” may display the page shown in FIG. 6. Selecting “notifications” may display the notifications center where any messages or reminders are collated. The user can also access their payment or insurance information. The lower section (1504-2) of the setting tab screen has a treatment preferences section via which the user can view their treatment details, e.g., the information entered during account creation, and view and adjust their injection routine and reminders. The setting tab screen also has a shared data section, in which the user can view and adjust the permissions they have granted for data to be shared with the application. This data may include the user device location, data from other applications, such as weather apps, data from activity monitors or physiological condition monitors, and data from connected devices, such as connected injection devices, connected inhalers, connected medicament storage devices, and connected sharps bins.

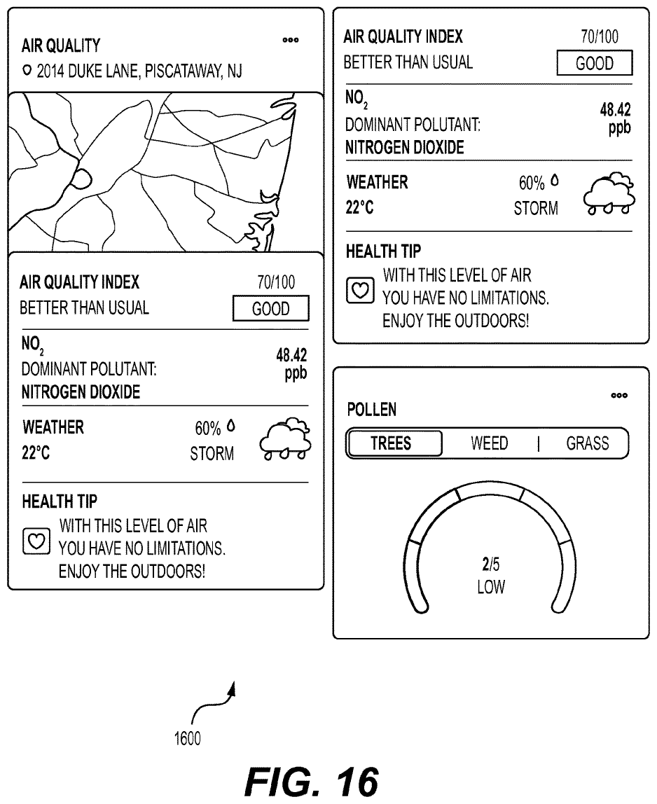

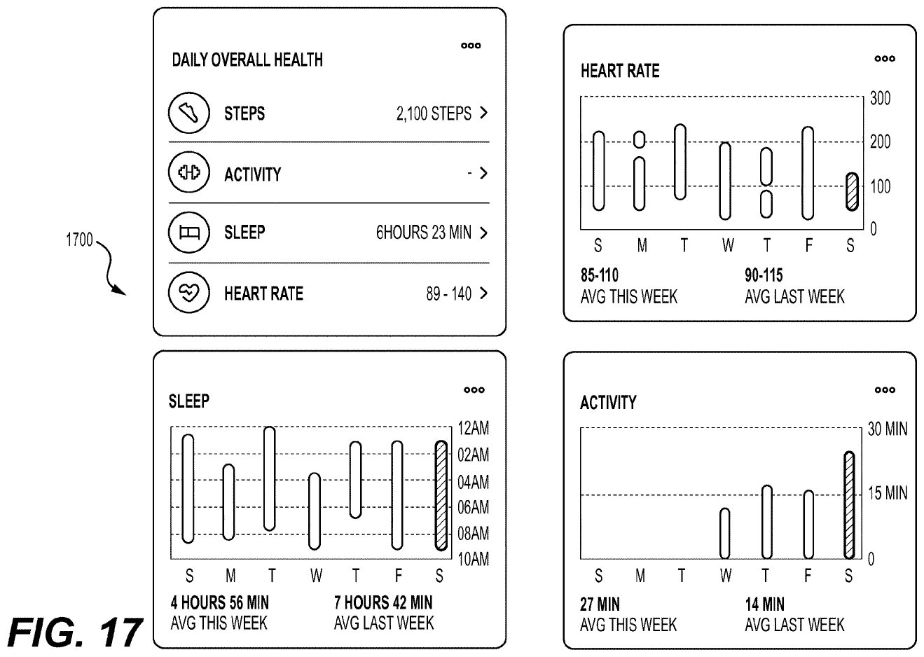

FIGS. 16 and 17 show exemplary information which may be gathered from external sources to be displayed in the application. FIG. 16 illustrates environmental information 1600 obtained from an environmental monitoring application. The environmental information 1600 may comprise current and forecast weather conditions, NO2 levels, an air quality index and pollen count, among other data. FIG. 17 shows user physiological and activity data 1700 collected by an activity tracker device. The user physiological and activity data 1700 may comprise a step count, heart rate, sleep time and length and a summarized level of overall activity, as well as other data.

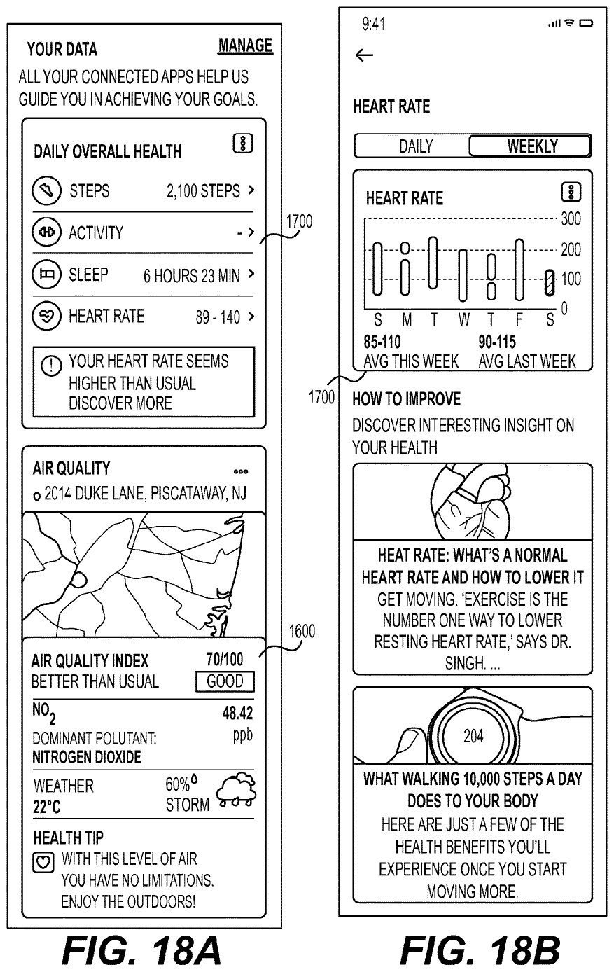

FIGS. 18a and 18b are exemplary screens showing how the environmental information 1600 and user physiological and activity data 1700 may be displayed within the application.

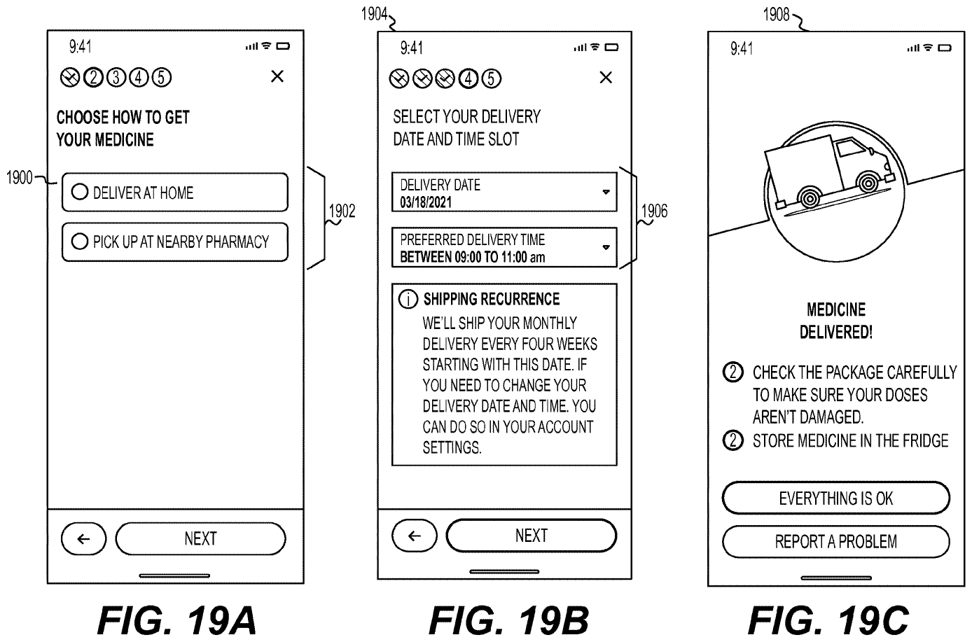

The application may also assist the user with medicament collection or delivery as illustrated in FIGS. 19a to 19c. FIG. 19a shows a delivery option screen 1900 comprising options 1902 for how the user would like to obtain their medicament. The options 1902 are to have the medicament delivered to their home or delivered to a pharmacy for their collection. If the user selects the pharmacy option, then a list of nearby pharmacies may be presented. If the user selects the home delivery option, then the delivery time selection screen 1904 of FIG. 19b may be displayed. The delivery time selection screen 1904 comprises options 1906 for selecting the date and time slot for delivery. The options 1906 may be in the form of drop down menus. Having selected a delivery time slot, the delivery confirmation screen 1908 of FIG. 19c may be shown. The delivery confirmation screen 1908 provides confirmation to the user that their medicament will be delivered. It may also provide information relating to receiving and/or storing the medicament, for example a reminder that the medicament should be placed in the fridge when received. The delivery confirmation screen 1908 may also be displayed at the time when the user receives their medicament and may comprise selectable button for reporting either safe reception of the medicament, or that there is some problem with the medicament.

In addition, the application allows the user to set and edit a refill reminder. As a first step, the application may ask the user to specify how many doses of the medicament they have left. The application uses the user's dosing frequency determined during account creation and customization and the number of doses remaining to determine when the user will run out of doses. Next the application may ask the user to specify how many days before (or after) they run out of doses to display a reminder. If in the first step the user indicated that they had no doses remaining and the application determines that the days remaining before the next scheduled dose is less than the reminder period selected, the application may display a warning advising the user to contact their pharmacy directly to obtain a refill of their prescription.

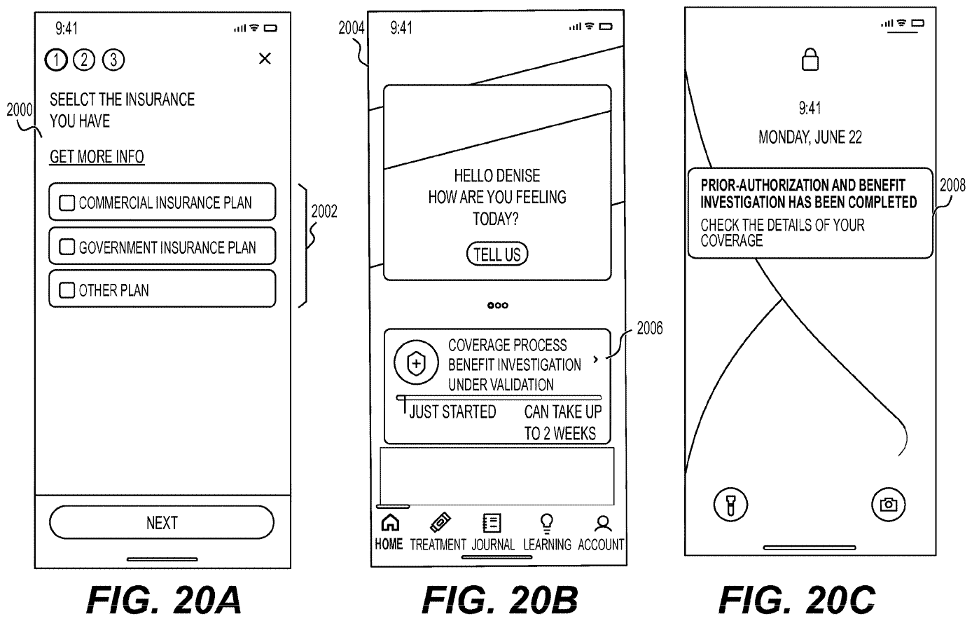

The application may also assist the user with linking their insurance details with the application and/or making an insurance claim to finance their prescription as illustrated in FIGS. 20a to 20c. FIG. 20a shows an insurance selection screen 2000 comprising options 2002 for allowing the user to indicate the type of insurance or other payment plan they have in relation to the first medicament. In the depicted embodiment, the options 2002 comprise a “commercial insurance plan”, a “Government insurance plan” or an “Other plan”. Having selected a particular insurance plan option, the program may collect the insurance plan details from the user in subsequent screens. After collecting the information, it may be communicated to an external system, for example the patient support portal illustrated in FIG. 11. While an investigation into the insurance plan is ongoing, the application may display a benefits notification 2006 informing the user that the investigation is underway. The benefits notification 2006 may be displayed on a home screen 2004 of the application as shown in FIG. 20b. once the benefits investigation is completed, the application may display a push notification 2008 on a lock screen of the user device informing the user of the outcome of the investigation. In this manner, the application can assist the user in many aspects of obtaining access to and financing the first medicament. The insurance selection screen 2000 may be shown as part of the account creation process or at a later time.

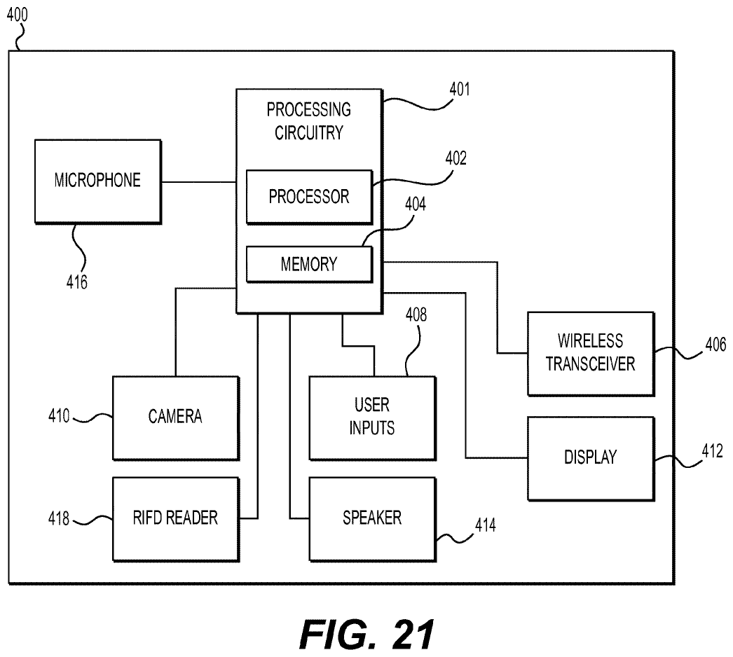

FIG. 21 illustrates schematically a user device 400 according to some embodiments. The user device 400 may be a mobile phone or tablet computer. The user device 400 is an example of a wireless communication device. The user device may alternatively be referred to as a communication device, computer, computing device or mobile device and is not necessarily associated with a single user. The user device 400 is configured to communicate wirelessly with a communications network using a wireless communication protocol, for example but not exclusively 3GPP LTE and/or New Radio (NR) or WiFi (IEEE 802.11). The user device 400 may also be configured to communicate using Bluetooth, NFC, Zigbee, Ultra Wideband, IrDa or similar. The communications network may comprise one or more network nodes. The communications network may further be connected via a core network and/or an intermediate network to a host computer (not shown) which may be embodied in the hardware and/or software of a standalone server, a cloud-implemented server or a distributed server. The user device 400 and the host computer may be configured to communicate data.

The user device 400 may comprise hardware that includes processing circuitry 401. The processing circuitry 401 may comprise a processor 402 and a memory 404. The user device 400 may also comprise a wireless transceiver 406, user inputs 408, a display 412, a camera 410, a microphone 416, and RFID reader 418 and a speaker 414. The wireless transceiver 406 may be configured to set up and maintain a wireless connection to a network node. The wireless transceiver 406 may comprise one or more radio transmitters and one or more radio receivers. The display may be a touch sensitive display and may be based on capacitive or resistive sensing technology. The processing circuitry 401 may for instance include a microprocessor, a Digital Signal Processor (DSP), Application Specific Integrated Circuit (ASIC), Field Programmable Gate Array (FPGA) or the like. The processor 402 may be configured to read and/or write from the memory 404. The memory 404 may comprise a volatile and/or nonvolatile memory, for example a cache, RAM (Random Access Memory) and/or ROM (Read-Only Memory) etc.

The user device 400 may comprise software which is stored, for example, in memory 404. The software may be executable by the processing circuitry 401. The software may include an application. In some embodiments the host computer may communicate with the application. The application may request data from the host computer and/or provide user data to the host computer.

The processing circuitry 401 may be configured to perform or cause to be performed any of the methods described herein. In some embodiments, the software/program may include instructions that, when executed by the processing circuitry 401 cause the processing circuitry 401 to perform the methods described herein.

The memory 404 may comprise both a program memory storing program code (e.g. software or firmware) and main memory storing data. The processing circuitry 401 may be configured to execute the program code stored in the program memory and to read, write and delete data from the main memory. In some embodiments, the program code may be an application which can be downloaded and installed on the user device 400. The application may be a disease and treatment management and tracking tool for use by patients. The program memory may for instance be a Read-Only Memory (ROM), and the main memory may for instance be a Random Access Memory (RAM).

The user device 400 comprises one or more user inputs 408, for example a touchscreen, keypad or keyboard, accelerometer or gyroscope, mouse or microphone 416 for receiving voice commands. User device 400 may also comprise a camera 410 configured to capture images of a user and images of labels, codes and the like visible on the medicament administration devices, packaging or storage solutions. The user device 400 may be configured to scan medicament administration devices (such as an injection device or inhaler) using a scanning device. The scanning device may refer to either the camera 410 or RFID reader 418. The term “scanning” as used in relation to the user device 400 may refer to use of either of these components to read information provided externally or internally on medicament administration devices.

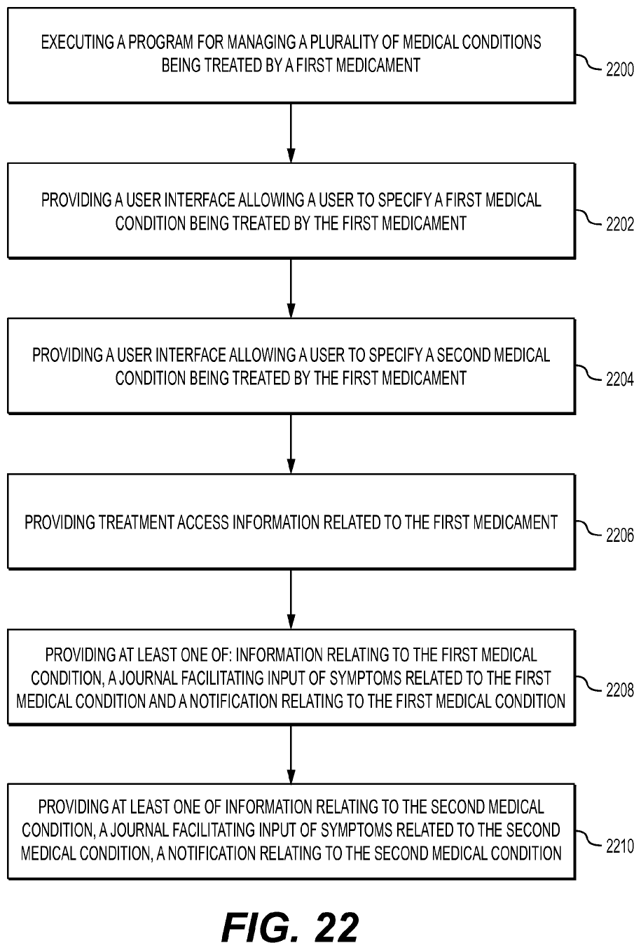

FIG. 22 is a flow chart of an example method for assisting a user with managing multiple health conditions being treated by a medicament. At operation 2200, a program (also referred to as an application) for managing a plurality of medical conditions being treated by a first medicament is executed on the computing apparatus/system. The method may be performed by computing apparatus/systems, such as those described in relation to FIG. 21. Upon execution, the program may perform a customization process relating to two or more health conditions of the user, as described below.

At operation 2202 a user interface allowing a user to specify a first medical condition being treated by the first medicament is provided, for example using the methods described in relation to FIGS. 2a to 2e. At operation 2204 a user interface allowing a user to specify a second medical condition being treated by the first medicament is provided, for example using the methods described in relation to FIGS. 2f to 2h.

Providing a user interface allowing the user to specify first and second medical conditions may comprise providing a single user interface display allowing the user to specify both the first medical condition being treated by the first medicament and the second medical condition being treated by the first medicament. The user interface may allow a user to specify further medicaments being used to treat the first medical condition, second medical condition or a further medical condition. In some other embodiments, the user interfaces for specifying the first and second (and further) medical conditions may be presented in separate or successive screens.

At operation 2206, treatment access information related to the first medicament is provided by the program. The treatment access information may comprise information on how to obtain a particular medicament and/or prescription information communicated to the program by an external source, such as a doctor or pharmacy. The treatment access information may comprise collection or delivery details for the first medicament. The treatment access information may comprise insurance and/or financial information related to obtaining the first medicament.

At operation 2208, at least one of: information relating to the first medical condition, a journal facilitating input of symptoms related to the first medical condition and a notification relating to the first medical condition is provided by the program is provided. The information relating to the first medical condition may comprise physiological data for the user and/or activity data for the user. The information relating to the first medical condition may comprise environmental information. Providing a notification relating to the first medical condition may comprise displaying a notification within the program or pushing a notification to be displayed on a lock screen or home screen of a user device running the program. The notification may provide a reminder to perform a certain action in relation to the first medical condition, such as attend an appointment, attend a call, administer a medicament or make a journal entry.

At operation 2210, at least one of: information relating to the second medical condition, a journal facilitating input of symptoms related to the second medical condition and a notification relating to the second medical condition is provided by the program is provided. The information relating to the second medical condition may comprise physiological data for the user and/or activity data for the user. The information relating to the second medical condition may comprise environmental information. Since the first medical condition and second medical condition are different, the relevant physiological data for the user and/or activity data for the user and the relevant environmental information may also be different.

Providing a notification relating to the second medical condition may comprise displaying a notification within the program or pushing a notification to be displayed on a lock screen or home screen of a user device running the program. The notification may provide a reminder to perform a certain action in relation to the second medical condition, such as attend an appointment, attend a call, administer a medicament or make a journal entry.

In additional to the operations described above with respect to FIG. 22, the computer implemented method may further comprise providing a dose support screen having a call-scheduling element configured to initiate a call booking process with a healthcare professional when selected. The computer implemented method may further comprise providing a pre-call instruction screen containing: instructions for preparing and/or administering the first medicament; and a call-initiating element configured to initiate a call by opening a phone application when selected. The computer implemented method may further comprise providing a post-call screen containing a body representation or model with multiple predefined areas for indicating the location of an injection.

In additional to the operations described above with respect to FIG. 22, the computer implemented method may further comprise providing a reminder system configured to: allow input of medication administration reminder parameters; and generate a reminder for display on a graphical user interface provided by the program for managing a plurality of medical conditions being treated by a first medicament or for display as a push notification on a locked screen of a user device.

In additional to the operations described above with respect to FIG. 22, the computer implemented method may further comprise pairing the program for managing a plurality of medical conditions being treated by a first medicament with one or more external devices and receiving user specific data from the one or more external devices. The user specific data may be physiological data and/or activity data. The computer implemented method may further comprise receiving environmental information and displaying the environmental information.

In additional to the operations described above with respect to FIG. 22, the computer implemented method may further comprise providing a prescription management system configured to: allow input of collection or delivery details for the first medicament; display status updates relating to collection or delivery details of the first medicament; and generate one or more push notifications relating to collection or delivery details of the first medicament.

In additional to the operations described above with respect to FIG. 22, the computer implemented method may further comprise providing a user interface facilitating the input of insurance and/or financial information related to accessing the first medicament; sending the insurance and/or financial information to a third party; and displaying an insurance and/or financial status notification related to accessing the first medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about 4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body. The drugs or medicaments contained in the drug delivery devices as described herein can be used for the treatment and/or prophylaxis of many different types of medical disorders.

As used herein, the terms “analogue” and “derivative” refers to a polypeptide which has a molecular structure which formally can be derived from the structure of a naturally occurring peptide, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. In particular, the term “derivative” refers to a polypeptide which has a molecular structure which formally can be derived from the structure of a naturally occurring peptide, in which one or more organic substituent (e.g., a fatty acid) is bound to one or more of the amino acids. Optionally, one or more amino acids occurring in the naturally occurring peptide may have been deleted and/or replaced by other amino acids, including non-codable amino acids, or amino acids, including non-codable, have been added to the naturally occurring peptide.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6R mAb (e.g., Sarilumab), and anti IL-4R mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof. An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1.-29. (canceled)

30. A computer-implemented method comprising:
executing a program for managing a plurality of medical conditions being treated by a first medicament;
providing a first user interface allowing a user to specify a first medical condition being treated by the first medicament;
providing a second user interface allowing the user to specify a second medical condition being treated by the first medicament;
providing treatment access information related to the first medicament;
providing at least one of information relating to the first medical condition, a journal facilitating input of symptoms related to the first medical condition, or a notification relating to the first medical condition; and
providing at least one of information relating to the second medical condition, a journal facilitating input of symptoms related to the second medical condition, or a notification relating to the second medical condition.

31. The computer-implemented method according to claim 30, wherein the first user interface is the same as the second user interface.

32. The computer-implemented method according to claim 30, wherein the first medical condition is Atopic Dermatitis and the second medical condition is Asthma.

33. The computer-implemented method according to claim 30, wherein at least one of the first user interface or the second user interface allows a user to specify further medicaments being used to treat the first medical condition, second medical condition or a further medical condition.

34. The computer-implemented method according to claim 30, further comprising at least one of the following:
pairing the program for managing a plurality of medical conditions being treated by a first medicament with one or more external devices and receiving user specific data from the one or more external devices, or
receiving environmental information and displaying the environmental information.

35. The computer-implemented method according to claim 34, wherein the user specific data is physiological data and/or activity data.

36. The computer-implemented method according to claim 30, further comprising:
determining that an authorization is required to customize the information related to the first medical condition and/or the information related to the second medical condition; and
presenting a user interface to obtain the authorization.

37. The computer-implemented method according to claim 36, wherein the authorization is to access a location of the user.

38. The computer-implemented method according to claim 30, further comprising providing a dose support screen having a call-scheduling element configured to initiate a call booking process with a healthcare professional when selected.

39. The computer-implemented method according to claim 38, further comprising providing a pre-call instruction screen containing:
instructions for preparing and/or administering the first medicament; and
a call-initiating element configured to initiate a call be opening a phone application when selected.

40. The computer-implemented method according to claim 39, further comprising providing a post-call screen containing a body representation with multiple predefined areas for indicating a location of an injection.

41. The computer-implemented method according to claim 30, further comprising providing a reminder system configured to:
allow input of medication administration reminder parameters; and
generate a reminder for display on a graphical user interface provided by the program for managing a plurality of medical conditions being treated by a first medicament or for display as a push notification on a locked screen of a user device.

42. The computer-implemented method according to claim 30, further comprising providing a prescription management system configured to:
allow input of collection or delivery details for the first medicament;
display status updates relating to collection or delivery details of the first medicament; and
generate one or more push notifications relating to collection or delivery details of the first medicament.

43. The computer-implemented method according to claim 30, further comprising:
providing a third user interface facilitating the input of insurance and/or financial information related to accessing the first medicament;
sending the insurance and/or financial information to a third party; and
displaying an insurance and/or financial status notification related to accessing the first medicament.

44. The computer-implemented method according to claim 30, further comprising:
scanning a code on an injection device in order to check an expiry date of medicament within the injection device; and
in response to a determination that the medicament within the injection device is expired, outputting a warning via the program.

45. The computer-implemented method according to claim 30, further comprising:
scanning a code associated with a new injection device or a new batch of injection devices;
launching the program and determining that the user is a registered user of the program; and
displaying a user interface asking the user to confirm that they have received a new injection device or batch of injection devices.

46. The computer-implemented method according to claim 45, further comprising:
in response to receiving a user input confirming that they have received the new injection device or batch of injection devices, causing a refill reminder in the program to be reset.

47. One or more non-transitory computer-readable storage media storing instructions that, when executed by at least one computer, cause the at least one computer to:
execute a program for managing a plurality of medical conditions being treated by a first medicament;
provide a user interface allowing a user to specify a first medical condition being treated by the first medicament;
provide a user interface allowing a user to specify a second medical condition being treated by the first medicament;
provide treatment access information related to the first medicament;
provide at least one of: information relating to the first medical condition, a journal facilitating input of symptoms related to the first medical condition and a notification relating to the first medical condition; and
provide at least one of information relating to the second medical condition, a journal facilitating input of symptoms related to the second medical condition, a notification relating to the second medical condition.

48. The one or more non-transitory computer-readable storage media of claim 47, wherein the instructions, when executed by the at least one computer, cause the at least one computer to perform at least one of the following:
providing a single user interface display allowing the user to specify both the first medical condition being treated by the first medicament and the second medical condition being treated by the first medicament, or
receiving environmental information and display the environmental information.

49. The one or more non-transitory computer-readable storage media of claim 47, wherein the instructions, when executed by the at least one computer, cause the at least one computer to:
determine that an authorization is required to customize the information relating to the first medical condition and/or the information relating to the second medical condition; and
present a user interface to obtain the authorization.

50. The one or more non-transitory computer-readable storage media of claim 49, wherein the authorization is to access a location of the user.

51. A user device comprising one or more processors and one or more storage devices storing instructions that, when executed by the one or more processors, cause the one or more processors to:
execute a program for managing a plurality of medical conditions being treated by a first medicament;
provide a first user interface allowing a user to specify a first medical condition being treated by the first medicament;
provide a second user interface allowing a user to specify a second medical condition being treated by the first medicament;
provide treatment access information related to the first medicament;
provide at least one of: information relating to the first medical condition, a journal facilitating input of symptoms related to the first medical condition and a notification relating to the first medical condition; and
provide at least one of information relating to the second medical condition, a journal facilitating input of symptoms related to the second medical condition, a notification relating to the second medical condition.

52. The user device of claim 51, wherein the instructions, when executed by the one or more processors, further cause the one or more processors to:
pair the program for managing a plurality of medical conditions being treated by a first medicament with one or more external devices and receive user specific data from the one or more external devices.

53. The user device of claim 52, wherein the user specific data is physiological data and/or activity data.

54. The user device of claim 51, wherein the instructions, when executed by the one or more processors, further cause the one or more processors to:
control a camera of the user device to scan a code on an injection device;
check an expiry date of medicament within the injection device based on the scanned code; and
when it is determined that the medicament within the injection device is expired, output a warning via the program.

55. The user device of claim 51, wherein the instructions, when executed by the one or more processors, further cause the one or more processors to:
control a camera of the user device to scan a code associated with a new injection device or a new batch of injection devices;
launch the program and determine that the user is a registered user of the program; and
display a user interface asking the user to confirm that they have received a new injection device or batch of injection devices.

56. The user device of claim 55, wherein the instructions, when executed by the one or more processors, further cause the one or more processors to:
in response to receiving a user input confirming that they have received the new injection device or batch of injection devices, cause a refill reminder in the program to be reset.
